(19) United States
(12) Patent Application Publication
(10) Pub. No.: US 2005/0075334 A1
(43) Pub. Date: Apr. 7, 2005

(54) Novel compounds

(76) Inventors: Sarah King, Loughborough (GB); Simon Teague, Loughborough (GB); Yafeng Xue, Molndal (SE); Britt-Marie Swahn, Sodertalje (SE)

Correspondence Address: WHITE & CASE LLP;PATENT DEPARTMENT, 1155 AVENUE OF THE AMERICAS, NEW YORK, NY, 10036, US

(21) Appl. No.: 10/499,599

(22) PCT Filed: Dec. 18, 2002
(86) PCT No.: PCT/SE02/02374

(30) Foreign Application Priority Data
Dec. 19, 2001 (SE) 0104331.4

(51) Int. Cl. A61K 31/541; A61K 31/5377; A61K 31/496; A61K 31/4709; A61K 31/4184; C07D 417/14; C07D 413/14; C07D 43/14

(52) U.S. Cl. 514/227.8; 514/234.2; 514/254.06; 514/314; 514/394; 544/60; 544/139; 544/368; 546/176; 548/305.1

(57) ABSTRACT

The present invention relates to new compounds of formula (I) wherein R¹, R², R³, R⁴, R⁵ and A are defined as in formula (I), a process for their preparation and new intermediate prepared therein, pharmaceutical compositions containing said therapeutically active compounds and to the use of said active compounds in therapy, especially in the treatment of c-Jun N-terminal kinase (JNK) mediated conditions in mammals, particularly Alzheimer's Disease.

### FIELD OF THE INVENTION

The present invention relates to new compounds of formula (I), as a free base, salts, solvates or solvates of salts thereof, to pharmaceutical formulations containing said compounds and to the use of said compounds in therapy. The present invention further relates to processes for the preparation of compounds of formula (I) and to new intermediates used in the preparation thereof.

### BACKGROUND TO THE INVENTION

Cells respond to and are regulated by their environment. Cell surface receptors are one of the most important means by which cells receive information from extra cellular signals. These receptors transmit information into the cell where it is propagated by activation or suppression of overlapping biochemical pathways. Protein kinases and phosphatases are important components of such intracellular signalling pathways as they allow the information to be cascaded to numerous effector molecules as well as giving amplification of the signal. Often complex arrays of pathways are activated by a given receptor leading to a coordinated cellular response. Analysis of these pathways in normal and diseased states has received considerable attention and it is now well accepted that aberrant or dysfunctional intracellular signalling contributes to the pathology of many disease states.

The MAP kinase signalling pathways are activated by engagement of a number of cell surface receptors. One of these pathways, the JNK pathway (named after one of the key enzymes in the pathway, c-jun N-terminal kinase or JNK) is activated specifically by stress or pro-inflammatory cytokines. Activators include LPS, the cytokines tumour necrosis factor (TNFα) and Interleukin-1 (IL1), osmotic shock, chemical stress and UV radiation (Cohen, P. Trends in Cell Biol. 7:353-361 1997). Targets of the JNK pathway include a number of transcription factors, such as but not exclusivelly c-jun and ATF-2 (Whitmarsh, A. and Davis, R. J. Mol. Med. 74:589-607 1998). These transcription factors bind as homo and heterodimers with AP-1 and AP-1 like sites in the promoters of a number of genes resulting in new gene expression (Karin, M. et al. Curr. Opin. Cell. Biol. 9:240-246 1997). Positive effects on gene expression are not always the outcome of JNK activation. In T cells, the calcineurin targeting domain of NFATc1 contains a site for JNK activity which blocks nuclear accumulation of the transcription factor. (Chow, C et al. Mol Cell. Biol., 20:5227-5234 2000).

Three different genes; JNK1, JNK2 and JNK3, encode the JNK family of enzymes. Alternatively spliced forms of these genes can give rise to 10 distinct isoforms; four for JNK1, four for JNK2 and two for JNK3. (Gupta, S. et al EMBO J. 15:2760-2770 1996). JNK1 and JNK2 are ubiquitously expressed in human tissues whereas JNK3 is selectively expressed in the brain, heart and testis (Dong, C. et al. Science 270:14 1998). The kinase activity of JNK1 is increased by phosphorylation of 2 key residues in the activation loop of the enzyme, Thr 183 and Tyr 185. Full activation depends on phosphorylation at both residues although reduced activity is seen if either residue is phosphorylated in isolation (Fleming Y. et al Biochem. J. 352:1451-54, 2000). The Thr-Pro-Tyr activation motif is conserved in all JNK isoforms and is homologous to the Thr-X-Tyr motif found in the activation loop of a related MAPkinase, p38 and the Thr-Y-Tyr in Erk 1 and Erk 2. In the case of p38, phosphorylation of the activation loop causes a conformational change in the protein exposing the ATP binding pocket. Although this method of activation has not been confirmed for JNK, a similar process is thought to take place.

To date, only two related enzymes, MKK4 and MKK7, are known to be able to phosphorylate JNK. MKK4 (also known as JNKK1) preferentially phosphorylates Tyr 185 although it is able to also phosphorylate Thr 183. (Lawler, S. et al Current Biology 8:1387-1390 1998). In vitro, MKK4 can also phosphorylate p38 although it is not certain whether this occurs in vivo (ref). In contrast, MKK7 (also known as JNKK2) can only phosphorylate Thr 183 (Lawler, S. et al Current Biology 8:1387-1390 1998). No other target for MKK7 has yet been found and it appears to be a JNK specific activator. A number of MAPKKK's have been reported to be able to activate JNK through activation of either MKK4 or MKK7, including MEKK1, MEKK2, TAK1, MLK. Selectivity of JNK signalling is most likely achieved through the specific interaction of the signalling components facilitated by the use of scaffold proteins. One such scaffold protein, JNK interacting protein 1 (JIP-1), selectively brings together JNK1, MKK7 and MLK (Yasuda, J. et al Mol. Cell. Biol. 19:7245-7254 1999).

JNK's 1, 2 and 3 have been selectively knocked out in mice both singulary and in combination by both gene deletion and/or transgenic expression of dominant negative forms of the kinases (Dong, C. et al Science 282:2092-2095 1998; Yang, D. et al Immunity 9:575-585 1998; Dong, C., et al Nature 405:91-94 2000; Yang, D. et al Nature 389:865-870 1997). Mice with targeted disruption of the JNK3 gene develop normally and are protected from excitotoxin induced apoptosis of neurones. This finding suggests that specific inhibitors of JNK 3 could be effective in the treatment of neurological disorders characterised by cell death such as Alzheimer's disease and stroke. Mice disrupted in either JNK 1 or 2 also develop normally. Peripheral T cells from either type of mice can be activated to make IL2 but in both cases, there is a defect in Th1 cell development. In the case of JNK1 −/− mice, this is due to an inability to make gamma interferon (a key cytokine essential for the differentiation of Th1 cells. In contrast, JNK2 −/− mice produce interferon gamma but are unable to respond to the cytokine. Similar defects in T cell biology (normal IL2 production but a block in Th1 cell differentiation) are seen in T cells disrupted in the MKK7 gene confirming this role for the JNK pathway in T cell differentiation (Dong, C., et al Nature 405:91-94 2000). MKK4 −/−mice have more profound defects in T cell activation and are blocked in IL2 production (Nishina, H. et al J. Exp. Med. 186:941-953 1997). However, in the light of the phenotype seen in the JNK−/− mice, this may be due to disruption of other roles of MKK4. Mice disrupted in both JNK1 and JNK 2 die in utero indicating an important role of JNK enzymes in development. However, T cells from the double knock out mice resemble T cells lacking MKK7. These data confirm an important role for the JNK pathway in T cell biology.

Activation of the JNK pathway has been documented in a number of disease settings, suggesting that specific inhibition of JNK activity could provide an effective therapy. Strong validation for a role for JNK in neurological disease has been obtained from the JNK3 deficient mice, as discussed above. Sinovial fibroblasts isolated from the joints of patients with rheumatoid arthritis have constitutively activated JNK activity and produce MMP's without any additional stimulation. They also respond strongly to pro-inflammatory stimuli such as TNFα or IL1 to increased further MMP expression (Han, Z et al. J Pharmacol Exp. Ther. 291:124-130 1999; Okamoto, K. et al. Arth. & Rheum. 40:919-926 1997). MMP expression can also be blocked by an inhibitor of JNK enzyme activity. This same inhibitor will block joint destruction in experimentally induced arthritis in mice (Han, Z. et al. J. Clin. Invest. 108:73-81 2001) providing strong support that the use of selective Compounds of formula (I) could be of benefit in human disease. It is likely that this protective effect is due to a blockade in MMP gene expression as a number of the MMP genes are under the control of AP-1 elements in their upstream promoter regions. Indeed, inducible expression of MMP3,9 and 13 are have been shown to be regulated through activation of JNK and AP-1 (Gum, R et al. Oncogene 14:1481-1493 1997).

JNK also plays a major role in apoptosis of cells (Davis R J. Cell. 103:239-252 2000). JNK is essential for U.V induced apoptosis through the cytochrome C mediated pathway (Tournier, C. et al Science 288:870-874 2000). Ischemia and ischemia coupled with reperfusion as well as restricted blood flow itself has been shown to be accompanied by activation of JNK. Cell death can be prevented with dominant negative forms of JNK transfected into cells demonstrating a potential utility for JNK in conditions characterised by stress induced apoptosis.

Activation of the JNK pathway has been observed in a number of human tumours and transformed cell lines (Davis R J. Cell. 103:239-252 2000). Indeed, one of the major targets of JNK, c-jun, was originally identified as an oncogene indicating the potential of this pathway to participate in unregulated cell growth. JNK also regulates phosphorylation of p53 and thus modulate cell cycle progression (Chen T. et al Mol. Carcinogenesis 15:215-226 1996). Inhibition of JNK may therefore be beneficial in some human cancers.

Based on current knowledge JNK signalling, especially JNK3, has been implicated in areas of apoptosis driven neurodegenerative diseases such as Alzheimer's disease, Parkinson's disease, ALS, epilepsy and seizures, Huntington's disease, traumatic brain injury, as well as ischemic and haemorrhaging stroke.

Thus there is a high unmet medical need for JNK specific inhibitors useful in treating the various conditions associated with JNK activation.

### DISCLOSURE OF THE INVENTION

The object of the present invention is to provide compounds having an inhibiting effect on JNK as well as having a good bioavailability.

The present invention provides a compound of formula (I) 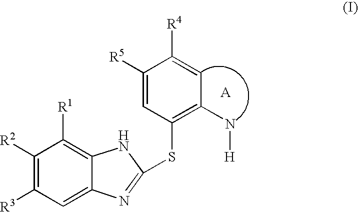
wherein:
R¹ is hydrogen, CHCHR⁶, CCR⁶, CO₂R⁷, NHCOR⁷, CN or halogen;
R² is hydrogen, halogen, CN, OC₁₋₆ alkyl, or a 5- or 6-membered heterocyclic ring containing 1, 2 or 3 heteroatoms selected independently from N, O and S;
R³ is hydrogen or halogen;
R⁴ is hydrogen, OH, NH₂, NO₂ or NHR⁸;
R⁵ is hydrogen, COR⁹, CHO, CH₂OR¹⁰, OH, OC₁₋₆ alkyl, NH₂, NR¹⁰R¹¹, NHCONR¹⁰R¹¹, NHCOR¹⁰, CONR¹²R¹³, CONHR⁷, R⁷, or
R⁴ and R⁵ together with the carbon atoms to which they are attached form a 5-, 6- or 7-membered lactam ring;
R⁶ is C₁₋₆ alkyl, substituted with 1, 2 or 3 substituents selected independently from hydroxy, OC₁₋₆ alkyl, OC₁₋₆ alkylOH and NR¹²R¹³;
R⁷ is CONHOC₁₋₆ alkylOH, or C₁₋₆ alkyl optionally substituted with 1, 2, 3 or 4 substituents selected independently from hydroxy, OC₁₋₆alkyl and NR¹²R¹³, or
R⁷ is phenyl or a 5- or 6-membered heterocyclic ring containing 1, 2, 3 or 4 heteroatoms selected independently from N, O and S;
R⁸ is COR¹⁴ or (CH₂)ₙQ where n is 1, 2 or 3 and Q is NR¹⁵R¹⁶ or a 5-, 6- or 7-membered saturated heterocyclic ring containing 1, 2 or 3 heteroatoms selected independently from N,
O and S, at least one of which must be N, with the exception that n can only be 1 when Q is a heterocyclic ring not linked through N;
R⁹ is hydroxy or OC₁₋₆ alkyl, which may be substituted with a 5- or 6-membered heterocyclic ring containing 1, 2 or 3 heteroatoms selected independently from N, O and S;
R¹⁰ and R¹¹ are selected independently from hydrogen and C₁₋₆ alkyl optionally substituted with 1, 2 or 3 substituents selected independently from hydroxy, OC₁₋₆ alkyl and NR¹²R¹³;
R¹² and R¹³ are selected independently from hydrogen and C₁₋₆ alkyl or R¹² and R¹³ form together a 5- or 6-membered heterocyclic ring containing 1, 2 or 3 heteroatoms selected independently from N, O and S and said ring may optionally be substituted with one or more substituents selected independently from hydroxy, oxo, C₁₋₆alkyl, OC₁₋₄ alkyl and C₁₋₆ alkylOH;
R¹⁴ is hydrogen or C₁₋₆ alkyl;
R¹⁵ and R¹⁶ are selected independently from hydrogen and C₁₋₆ alkyl;
A is a 5- or 6-membered saturated or a 5- or 6-membered aromatic ring optionally containing one or more heteroatoms selected independently from N, O and S, containing one or more C═O groups, and said ring may optionally be substituted with one or more substituents selected independently from C₁₋₆ alkyl, SO₂C₁₋₆ alkyl, SOC₁₋₆ alkyl, CO₂H and CHO, as a base, salts, solvates or solvates of salts thereof.

One aspect of the invention relates to compounds of formula (I), wherein:
R² is hydrogen, halogen, CN, or a 5- or 6-membered heterocyclic ring containing 1, 2 or 3 heteroatoms selected independently from N, O and S;
R⁵ is hydrogen, COR⁹, CHO, CH₂OR¹⁰, OH, OC₁₋₆ alkyl, NH₂, NR¹⁰R¹¹, NHCONR¹⁰OR¹¹, NHCOR¹⁰ or
R⁴ and R⁵ together with the carbon atoms to which they are attached form a 5-, 6- or 7-membered lactam ring;
R⁶ is C₁₋₆ alkyl, substituted with 1, 2 or 3 substituents selected independently from hydroxy, OC₁₋₆ alkyl and NR¹²R¹³;
R⁹ is hydroxy or OC₁₋₆ alkyl; and
R¹² and R¹³ are selected independently from hydrogen and C₁₋₆ alkyl.

In another aspect of the invention R¹ is hydrogen, CN, CO₂CH₃, CO₂CH₂CH₃, CO₂CH₂CH₂OH, halogen or NHCOR⁷, wherein R⁷ is furyl or phenyl.

In a further aspect of the invention R² is hydrogen or chloro.

In yet another aspect of the invention R³ is hydrogen.

In another aspect of the invention R⁴ is hydrogen, NH₂, NO₂ or NHR⁸, wherein R⁸ is CH₂-pyrrolidine.

In yet another aspect of the invention R⁴ is NHR⁸, wherein R⁸ is CH₂-piperidine.

In one aspect of the invention R⁵ is hydrogen or COR⁹, wherein R⁹ is hydroxy.

In another aspect of the invention R⁵ is CONR¹²R¹³, CONHR⁷ or R⁷.

In one aspect of the invention R⁷ is C₁₋₆alkyl optionally substituted with 1, 2 or 3 substituents selected independently from hydroxy, OC₁₋₆alkyl and NR¹²R¹³, such as N-diC₁₋₆ alkyl, or

R⁷ is phenyl or a 5- or 6-membered heterocyclic ring containing 1, 2, 3 or 4 heteroatoms selected independently from N, O and S.

In a further aspect of the invention R⁷ is CONHOC₁₋₆alkylOH, or C₁₋₆ alkyl optionally substituted with hydroxy, or

R⁷ is phenyl or a 5- or 6-membered heterocyclic ring containing 1, 2, 3 or 4 heteroatoms selected independently from N and O.

In yet a further aspect of the invention R⁷ include CH₂CH₂OH, CH₂OH and CH(CH₂OH)CH₂OH.

In another aspect of the invention R⁷ is furyl, piperidinyl, phenyl or CH₂CH₂OH. Examples of suitable 5- or 6-membered heterocyclic rings include tetrazolyl, thienyl, furyl or piperidinyl.

In yet another aspect of the invention A is a 5- or 6-membered saturated or a 5- or 6-membered aromatic ring optionally containing one or more heteroatoms selected independently from N, O and S; containing one or more C═O groups.

In one aspect of the invention, group A, together with the phenyl group to which it is attached, forms a bicyclic group, including the groups below: 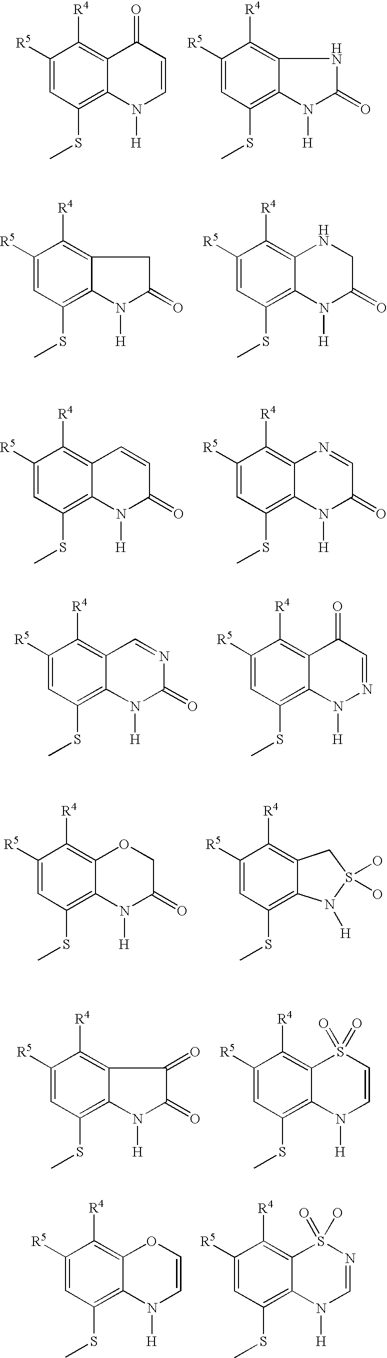 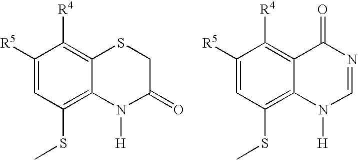
wherein R⁴ and R⁵ are as defined in formula (I).

One aspect of the invention relates to compounds of formula (I), wherein group A, together with the phenyl group to which it is attached, forms a bicyclic group of formula (A) or (B): 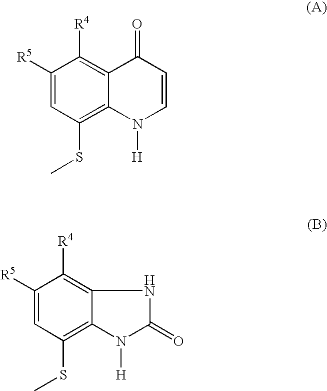
wherein R⁴ and R⁵ are as defined in formula (I).

Another aspect of the invention refers to compounds, which are
8-(1H-benzimidazol-2-ylsulfanyl)-4-(1H)-quinolinone,
8-[(5-chloro-1H-benzimidazol-2-yl)sulfanyl]4(1H)-quinolinone,
2-(4-oxo-1,4-dihydro-quinolin-8-ylsulfanyl)-1H-benzoimidazole-4-carboxylic acid methyl ester,
8-[(5-chloro-1H-benzimidazol-2-yl)sulfanyl]-5-nitro-4(1H)-quinolinone,
5-amino-8-[(5-chloro-1H-benzimidazol-2-yl)sulfanyl]4(1H)-quinolinone,
8-[(5-chloro-1H-benzimidazol-2-yl)sulfanyl]-5-{[(2r)-pyrrolidinylmethyl]amino)}-4(1H)-quinolinone,
8-[(5-chloro-1H-benzimidazol-2-yl)sulfanyl]-5-[(4-piperidinylmethyl)amino]-4(1H)-quinolinone,
5-chloro-2-[(4-oxo-1,4-dihydroquinolin-8-yl)thio]-1H-benzimidazole-4-carbonitrile, methyl 5-chloro-2-[(4-oxo-1,4-dihydroquinolin-8-yl)thio]-1H-benzimidazole-4 carboxylate,
N-{5-chloro-2-[(4-oxo-1,4-dihydroquinolin-8-yl)thio]-1H-benzimidazol-4-yl}benzamide,
N-{5-chloro-2-[(4-oxo-1,4-dihydroquinolin-8-yl}thio]-1H-benzimidazol-4-yl-2-furamide,
4[(5-chloro-1H-benzimidazol-2-yl)thio]-1,3-dihydro-2H-benzimidazol-2-one,
7-[(5-chloro-1H-benzimidazol-2-yl)thio]-2-oxo-2,3-dihydro-1H-benzimidazole-5-carboxylic acid,
7-{[5-chloro-4-(methoxycarbonyl)-1H-benzimidazol-2-yl]thio}-2-oxo-2,3-dihydro-1H-benzimidazole-5-carboxylic acid,
7-({5-chloro-4-[(2-hydroxyethoxy)carbonyl]-1H-benzimidazol-2-yl}thio)-2-oxo-2,3-dihydro-1H-benzimidazole-5-carboxylic acid, methyl 7-[(5-chloro-1H-benzimidazol-2-yl)thio]-2-oxo-2,3-dihydro-1H-benzimidazole-5-carboxylate, ethyl 7-[(5-chloro-1H-benzimidazol-2-yl)thio]-2-oxo-2,3-dihydro-1H-benzimidazole-5-carboxylate,
2-morpholin-4-ylethyl 7-[(5-chloro-1H-benzimidazol-2-yl)thio]-2-oxo-2,3-dihydro-1H-benzimidazole-5-carboxylate,
7-[(5-chloro-6-fluoro-1H-benzimidazol-2-yl)thio]-2-oxo-2,3-dihydro-1H-benzimidazole-5-carboxylic acid,
7-[(5-methoxy-1H-benzimidazol-2-yl)thiol]-2-oxo-2,3-dihydro-1H-benzimidazole-5-carboxylic acid,
7-[(5-bromo-1H-benzimidazol-2-yl)thio]-2-oxo-2,3-dihydro-1H-benzimidazole-5-carboxylic acid,
7-[(4-bromo-6-fluoro-1H-benzimidazol-2-yl)thio]-2-oxo-2,3-dihydro-1H-benzimidazole-5-carboxylic acid,
7-[(5-chloro-1H-benzimidazol-2-yl)thio]-N-[2-(4-morpholin-4-ylethyl]-2-oxo-2,3-dihydro-1H-benzimidazole-5-carboxamide,
7-[(5-chloro-1H-benzimidazol-2-yl)thio]-N-[3-dimethylamino)ethyl]-2-oxo-2,3-dihydro-1H-benzimidazole-5-carboxamide,
4-[(5-chloro-1H-benzimidazol-2-yl)thio]-6-[(4-methylpiperazin-1-yl)carbonyl]-1,3-dihydro-2H-benzimidazol-2-one,
7-[(5-chloro-1H-benzimidazol-2-yl)thio]-N-methyl-2-oxo-2,3-dihydro-1H-benzimidazole-5-carboxamide,
7-[(5-chloro-1H-benzimidazol-2-yl)thio]-N-(2-hydroxyethyl)-2-oxo-2,3-dihydro-1H-benzimidazole-5-carboxamide,
4[(5-chloro-1H-benzimidazol-2-yl)thio]-6-(piperazin-1-ylcarbonyl)-1,3-dihydro-2H-benzimidazol-2-one dihydrochloride,
7-[(5-chloro-1H-benzimidazol-2-yl)thio]-2-oxo-2,3-dihydro-1H-benzimidazole-5-carboxamide,
7-[(5-chloro-H-benzimidazol-2-yl)thio]-N-[2-(2-hydroxyethoxy)ethyl]-2-oxo-2,3-dihydro-1H-benzimidazole-5-carboxamide,
4-[(5-chloro-1H-benzimidazol-2-yl)thio]-6-{[4-(2-hydroxyethyl)piperazin-1-yl]carbonyl}-1,3-dihydro-2H-benzimidazol-2-one,
7-[(5-chloro-1H-benzimidazol-2-yl)thio]-2-oxo-N4-piperidin-4-yl-2,3-dihydro-1H-benzimidazole-5-carboxamide dihydrochloride,
N-(3-aminopropyl)-7-[(5-chloro-1H-benzimidazol-2-yl)thio]-2-oxo-2,3-dihydro-1H-benzimidazole-5-carboxamide dihydrochloride,
7-[(5-chloro-1H-benzimidazol-2-yl)]thio-2-oxo-N-[3-(2-oxopyrrolidin-1-yl)propyl]-2,3-dihydro-1H-benzimidazole-5-carboxamide, and
4[(5-chloro-1H-benzimidazol-2-yl)thio]-6-(hydroxymethyl) 1,3-dihydro-2H-benzimidazol-2-one,
   as a base, salts, solvates or solvates of salts thereof.

The present invention further provides a compound of formula (I) 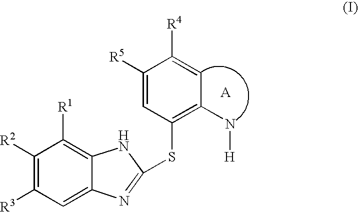
wherein:
R¹ is hydrogen, CO₂R⁷, NHCOR⁷ or halogen;
R² is hydrogen or halogen;
R³ is hydrogen or halogen;
R⁴ is hydrogen, NH₂ or NO₂;
R⁵ is hydrogen;
R⁷ is C₁₋₆ alkyl optionally substituted with 1, 2, 3 or 4 substituents selected independently from hydroxy;
A is a 5-membered aromatic ring containing one or more heteroatoms selected independently from N and O; and said ring may optionally be substituted with one or more substituents selected independently from C₁₋₆ alkyl, SO₂C₁₋₆ alkyl, SOC₁₋₆ alkyl and CHO, as a base, salts, solvates or solvates of salts thereof.

Substituents on A can be the same or different, and can be attached to any suitable carbon or nitrogen atom of ring A. In an aspect of the invention group A, together with the phenyl group to which it is attached, forms a bicyclic group, including the groups below: 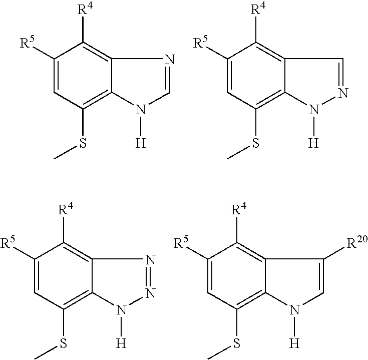
wherein R²⁰ is SOCH₃, SO₂CH₃, CH₃, CO₂H or CHO.

One aspect of the invention relates to bicyclic group (C) 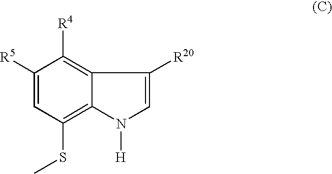
wherein R⁴ and R⁵ are as defined in formula (I) and R²⁰ is SOCH₃, SO₂CH₃, CH₃, CO₂H or CHO.

The invention further relates to compounds, which are
7-[(5-chloro-1H-benzimidazol-2-yl)thio]-3-(methylsulfonyl)-1H-indol-4-amine, methyl 5-chloro-2-{[3-methylsulfonyl)-1H-indol-7-yl]thiol}-1H-benzimidazole-4-carboxylate,
5-chloro-2-[(3-methyl-4-nitro-1H-indol-7-yl)thio]-1H-benzimidazole,
7-[(5-chloro-1H-benzimidazol-2-yl)thio]-3-methyl-1H-indol-4-amine,
5-chloro-2-{[3-(methylsulfinyl)-4-nitro-1H-indol-7-yl]thio}-1H-benzimidazole,
7-[(5-chloro-1H-benzimidazol-2-yl)thio]-3-(methylsulfinyl)-1H-indol-4-amine,
4-amino-7-[(5-chloro-1H-benzimidazol-2-yl)thio]-1H-indole-3-carbaldehyde, and
7-[(5-chloro-1H-benzimidazol-2-yl)thio]-3-(methylsulfonyl)-1H-indole,
   as a base, salts, solvates or solvates of salts thereof.

One aspect of the invention relates to compounds of formula (I) wherein:
R¹ is hydrogen, CHCHR⁶, CCR⁶, CO₂R⁷, NHCOR⁷, CN or halogen;
R² is hydrogen, halogen, CN, or a 5- or 6-membered heterocyclic ring containing 1, 2 or 3 heteroatoms selected independently from N, O and S;
R³ is hydrogen or halogen;
R⁴ is hydrogen, OH, NH₂, NO₂ or NHR⁸;
R⁵ is hydrogen, COR⁹, CHO, CH₂OR¹⁰, OH, OC₁₋₆ alkyl, NH₂, NR¹⁰R¹¹, NHCONR¹⁰OR¹¹, NHCOR¹⁰, or
R⁴ and R⁵ together with the carbon atoms to which they are attached form a 5-, 6- or 7-membered lactam ring;
R⁶ is C₁₋₆ alkyl, substituted with 1, 2 or 3 substituents selected independently from hydroxy, OC₁₋₆ alkyl and NR¹²R¹³;
R⁷ is C₁₋₆ alkyl optionally substituted with 1, 2 or 3 substituents selected independently from hydroxy, OC₁₋₆alkyl and NR¹²R¹³, or
R⁷ is phenyl or a 5- or 6-membered heterocyclic ring containing 1, 2 or 3 heteroatoms selected independently from N, O and S;
R⁸ is COR¹⁴ or (CH₂)ₙQ where n is 1, 2 or 3 and Q is NR¹⁵R¹⁶ or a 5-, 6- or 7-membered saturated heterocyclic ring containing 1, 2 or 3 heteroatoms selected independently from N,
O and S, at least one of which must be N, with the exception that n can only be 1 when Q is a heterocyclic ring not linked through N;
R⁹ is hydroxy or OC₁₋₆ alkyl;
R¹⁰ and R¹¹ are selected independently from hydrogen and C₁₋₆ alkyl optionally substituted with 1, 2 or 3 substituents selected independently from hydroxy, OC₁₋₆ alkyl and NR¹²R¹³;
R¹² and R¹³ are selected independently from hydrogen and C₁₋₆ alkyl;
R¹⁴ is hydrogen or C₁₋₆ alkyl;
R¹⁵ and R¹⁶ are selected independently from hydrogen and C₁₋₆alkyl;
A is a 5- or 6-membered saturated or aromatic ring optionally containing one or more heteroatoms selected from N, O and S, and further optionally containing one or more C═O groups, and said ring may optionally be substituted with one or more substituents selected independently from C₁₋₆ alkyl, SO₂C₁₋₆ alkyl, SOC₁₋₆ alkyl and CHO.

In the context of the present specification, unless otherwise indicated, an alkyl group whether alone or as part of another group may be linear or branched.

The term “C₁₋₆ alkyl” denotes a straight-chain or branched saturated aliphatic hydrocarbon having from 1 to 6 carbon atoms. Examples of said alkyl include, but are not limited to, methyl, ethyl, n-propyl, iso-propyl, n-butyl, iso-butyl, sec-butyl, t-butyl and straight- and branched-chain pentyl and hexyl.

In this specification, unless stated otherwise, the term “CHCH” refers to an alkenyl group. The term “alkenyl” includes both straight and branched chain alkenyl groups but references to individual alkenyl groups such as 2-butenyl are specific for the straight chain version only. Unless otherwise stated, the term “alkenyl” advantageously refers to chains with 2 to 5 carbon atoms, preferably 3 to 4 carbon atoms. Thus, the term “CHCHR⁶” refers to an alkenyl group substituted with R⁶.

In this specification, unless stated otherwise, the term “CC” refers to an alkynyl group. The term “alkynyl” includes both straight and branched chain alkynyl groups but references to individual alkynyl groups such as 2-butynyl are specific for the straight chain version only. Unless otherwise stated, the term “alkynyl” advantageously refers to chains with 2 to 5 carbon atoms, preferably 3 to 4 carbon atoms. Thus the term “CCR⁶” refers to an alkynyl group substituted with R⁶.

The term “halogen” includes fluoro, chloro, bromo and iodo groups.

The term “heterocyclic ring” denotes a 3- to 10-membered, aromatic, non-aromatic partially or completely saturated hydrocarbon group, which contains one or two rings and at least one heteroatom. Examples of said heterocycle include, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl, pyrrolidinyl, pyrrolidonyl, piperidinyl, piperazinyl, morpholinyl, oxazolyl, 2-oxazolidonyl or tetrahydrofuranyl.

Certain compounds of formula (I) may exist in stereoisomeric forms. It will be understood that the invention encompasses all geometric and optical isomers of the compounds of formula (I) and mixtures thereof including racemates.

The invention relates to any and all tautomeric forms of the compounds of formula (I).

The present invention relates to the use of compounds of formula (I) as hereinbefore defined as well as to salts thereof. Salts for use in pharmaceutical compositions will be pharmaceutically acceptable salts, but other salts may be useful in the production of the compounds of formula (I) and their pharmaceutically acceptable salts.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

The present invention also relates to compounds of formula (I), wherein the salts are pharmaceutically acceptable salts.

Medical Use

Surprisingly, it has been found that the compounds of the present invention, as a free base, salts, solvates or solvates of salts thereof, are well suited for inhibiting JNK. Accordingly, the compounds of the present invention are expected to be useful in the treatment of JNK-mediated condition, i.e. the compounds may be used to produce an inhibitory effect of JNK in mammals, including man, in need of such treatment.

The term “JNK-mediated condition”, as used herein means any disease or other deleterious condition in which JNK is known to play a role. Such conditions include, without limitation, inflammatory diseases, autoimmune diseases, destructive bone disorders, proliferative disorders, cancer, infectious diseases, neurodegenerative diseases, allergies, reperfusion/ischemia in stroke, heart attacks, angiogenic disorders, organ hypoxia, vascular hyperplasia, cardiac hypertrophy, thrombin-induced platelet aggregation, and conditions associated with prostaglandin endoperoxidase synthase-2.

Inflammatory diseases which may be treated or prevented by the compounds of this invention include, but are not limited to, acute pancreatitis, chronic pancreatitis, asthma, allergies and adult respiratory distress syndrome.

Autoimmune diseases which may be treated or prevented by the compounds of this invention include, but are not limited to, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis and graft vs. host disease.

Destructive bone disorders which may be treated or prevented by the compounds of this invention include, but are not limited to, osteoporosis, osteoarthritis and multiple myeloma-related bone disorder.

Proliferative diseases which may be treated or prevented by the compounds of this invention include, but are not limited to, acute myelogenous leukemia, chronic myelogenous leukemia, metastatic melanoma, Kaposi's sarcoma, multiple myeloma and HTLV-1 mediated tumorigenesis.

Angiogenic disorders which may be treated or prevented by the compounds of this invention include, but are not limited to, solid tumors, ocular neovasculization and infantile haemangiomas. Infectious diseases which may be treated or prevented by the compounds of this invention include, but are not limited to, sepsis, septic shock and Shigellosis. Viral diseases which may be treated or prevented by the compounds of this invention include, but are not limited to, acute hepatitis infection (including hepatitis A, hepatitis B and hepatitis C), HIV infection and CMV retinitis.

Neurodegenerative diseases which may be treated or prevented by the compounds of this invention include, but are not limited to, Alzheimer's disease, Parkinson's disease, amyotrophic lateral sclerosis (ALS), epilepsy, seizures, Huntington's disease, traumatic brain injury, ischemic and hemorrhaging stroke, cerebral ischemias and neurodegenerative disease such as apoptosis-driven neurodegenerative disease that may be caused by traumatic injury, acute hypoxia, ischemia or glutamate neurotoxicity.

“JNK-mediated conditions” also include ischemia/reperfusion in stroke, heart attacks, myocardial ischemia, organ hypoxia, vascular hyperplasia, cardiac hypertrophy, hepatic ischemia, liver disease, congestive heart failure, pathologic immune responses such as that caused by T cell activation and thrombin-induced platelet aggregation.

In addition, compounds of the instant invention may be capable of inhibiting the expression of inducible pro-inflammatory proteins. Therefore, other “JNK-mediated conditions” which may be treated or prevented by the compounds of this invention include edema, analgesia, fever and pain, such as neuromuscular pain, headache, cancer pain, dental pain and arthritis pain.

One embodiment of the invention relates to the use of the compounds of formula (I) in the treatment JNK mediated conditions selected from the group consisting of Alzheimer's disease, Parkinson's disease, ALS, epilepsy and seizures, Huntington's disease, traumatic brain injury, as well as ischemic and haemorrhaging stroke.

In a preferred embodiment the invention the condition is Alzheimer's Disease.

The present invention relates also to the use of the compound of formula (I) as defined hereinbefore, in the manufacture of a medicament for the treatment of JNK mediated condition and any other condition mentioned hereinbefore.

The present invention relates further to the use of the compound of formula (I) as defined hereinbefore, in the manufacture of a medicament for the treatment of Alzheimer's disease, Parkinson's disease, ALS, epilepsy and seizures, Huntington's disease, traumatic brain injury or haemorrhaging stroke.

The present invention relates to the use of the compound of formula (I) as defined hereinbefore, in the manufacture of a medicament for the treatment of Alzheimer's disease.

According to another embodiment, the invention provides a method of treatment of JNK mediated conditions and any other condition mentioned hereinbefore, comprising administering to a patient in need of such treatment, a therapeutically effective amount of the compound of formula (I).

In the context of the present specification, the term “therapy” and “treatment” also includes “prevention” unless there are specific indications to the contrary; The terms “treat”, “therapeutic” and “therapeutically” should be construed accordingly.

The term “patient”, as used herein, means an animal, preferably a human.

In this specification, unless stated otherwise, the term ‘inhibitor’ means a compound that by any means, partly or completely, blocks the transduction pathway leading to the production of a response by the ligand.

The term “condition”, unless stated otherwise, means any disorder and disease associated with JNK activity.

Non-Medical Use

In addition to their use in therapeutic medicine, the compounds of formula (I) as a free base, salts, solvates or solvates of salts thereof, are also useful as pharmacological tools in the development and standardisation of in vitro and in vivo test systems for the evaluation of the effects of inhibitors of JNK related activity in laboratory animals such as cats, dogs, rabbits, monkeys, rats and mice, as part of the search for new therapeutics agents.

Pharmaceutical Compositions

The present invention provides a compound of formula (I), or pharmaceutically acceptable salts thereof, as hereinbefore defined for use in therapy.

The compounds of formula (I) or pharmaceutical salts thereof maybe formulated into pharmaceutical compositions for administration to animals or humans.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection and infusion techniques.

Preferably, the compositions are administered orally, intraperitoneally or intravenously.

The pharmaceutical compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, may also be added. Examples of useful diluents include lactose and dried cornstarch. When aqueous suspensions are required for oral use, the active ingredient may be combined with emulsifying and suspending agents. If desired, certain sweetening, flavoring or coloring agents may also be added.

In general the above compositions may be prepared in a conventional manner using pharmaceutically acceptable excipients, diluents and/or inert carriers.

The amount of JNK inhibitor that may be combined with the carrier materials to produce a single dosage form will vary depending upon the host treated, the particular mode of administration. Preferably, the compositions should be formulated so that a dosage of between 0.01-100 mg/kg body weight/day of the inhibitor can be administered. It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Thus, the invention also relates to a pharmaceutical composition comprising as active ingredient a therapeutically effective amount of the compound of formula (I) in association with pharmaceutically acceptable excipients, diluents and/or inert carriers.

This pharmaceutical composition may be used in the treatment of JNK mediated conditions and any other condition mentioned hereinbefore.

An example of a pharmaceutical composition of the invention is an injectable solution containing a compound of the invention (4[(5-Chloro-1H-benzimidazol-2-yl)thio]-6 (piperazin-1-ylcarbonyl)-1,3-dihydro-2H-benzimidazol-2-one dihydrochloride), or a pharmaceutically acceptable salt thereof, as hereinbefore defined, and sterile water, and, if necessary, either sodium hydroxide or hydrochloride acid to bring the pH of the final composition to about pH 5, and optionally a surfactant to aid dissolution.

Liquid composition comprising a compound of formula (I), or a pharmaceutically acceptable salt thereof, dissolved in water.

| Liquid composition | mg/ml |
|---|---|
| 4-[(5-Chloro-1H-benzimidazol-2-yl)thio]-6- | 5.0% w/v |
| (piperazin-1-ylcarbonyl)-1,3-dihydro-2H- | |
| benzimidazol-2-one dihydrochloride | |
| Pure water | up to 100% |

Method of Preparation

The compounds of this invention may be prepared by methods known to those skilled in the art for analogous compounds, as illustrated by the general schemes below and by the preparative examples that follow.

An aspect of the invention relates to processes (a) and (b) for the preparation of compounds of formula (I) comprising of
(a) reacting a compound of formula (II): 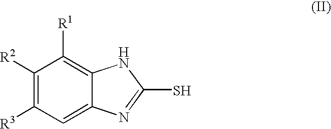
   wherein R¹, R² and R³ are as defined in formula (I) or are protected derivatives thereof, with a compound of formula (III): 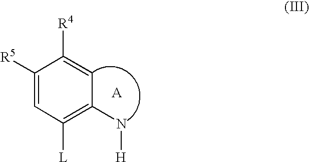
   wherein A, R⁴ and R⁵ are as defined in formula (I) or are protected derivatives thereof and L is a leaving group such as halogen, preferably fluoro. This reaction may be performed in an alcohol such as n-propanol or ethanol, for 10-48 hours at 90-100° C. in the presence of a base such as potassium hydroxid or sodium hydroxide; or
(b) reacting a compound of formula (IV): 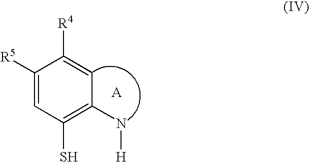
   wherein A, R⁴ and R⁵ are as defined in formula (I) or are protected derivatives thereof; with a compound of formula (V): 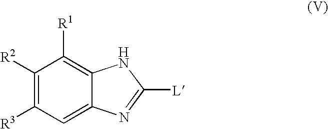
   wherein R¹, R² and R³ are as defined in formula (I) or are protected derivatives thereof and L′ is a leaving group such as SO₂ Me or halogen such as fluoro, chloro or bromo. This reaction may be performed in refluxing alcohol, such as a lower alcohol e.g. isopropanol or sec-butyl alcohol, for 2-48 hours.

Intermediate compounds of formula (II) can be prepared using known chemistry, for example according to the scheme below: where the nitro group can be reduced with hydrogen in the presence of a catalyst such as Pd or Pt on carbon. The diamino compound is reacted with carbon disulfide in an inert solvent such as dimethyl formamide (Org. Synth. 30, 1950, 56) to yield intermediate (II)

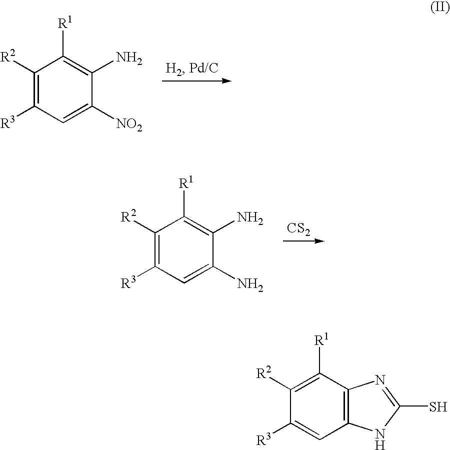

Intermediate compounds of formula (III) can be prepared using known chemistry, for example according to the scheme below:

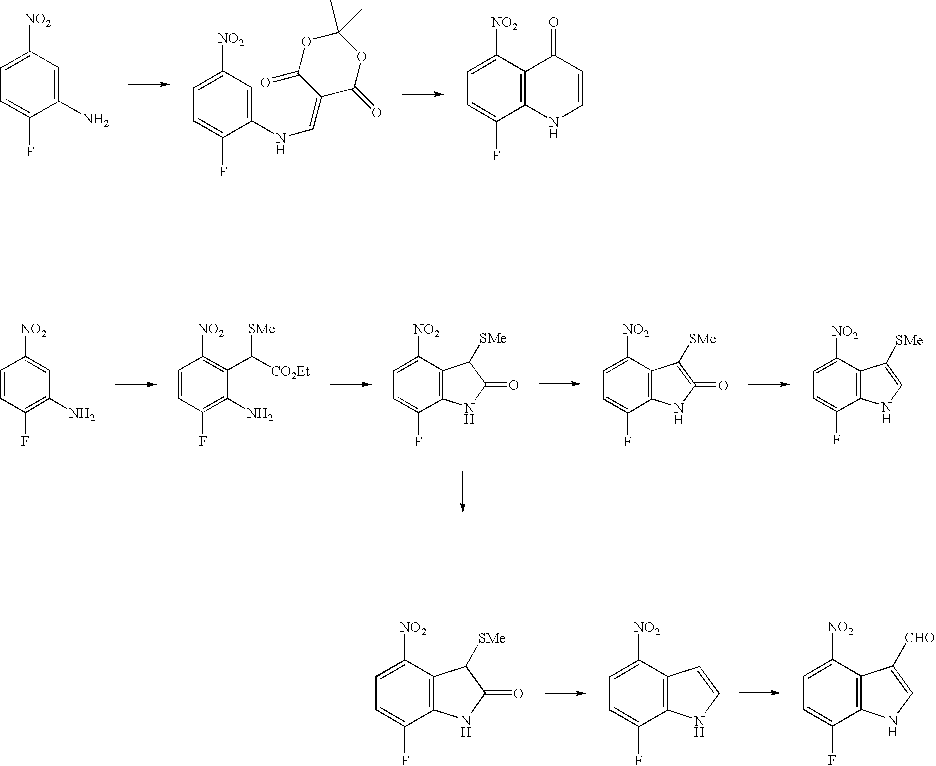

Intermediate compounds of formula (IV) can be prepared using known chemistry, for example according to the scheme below:

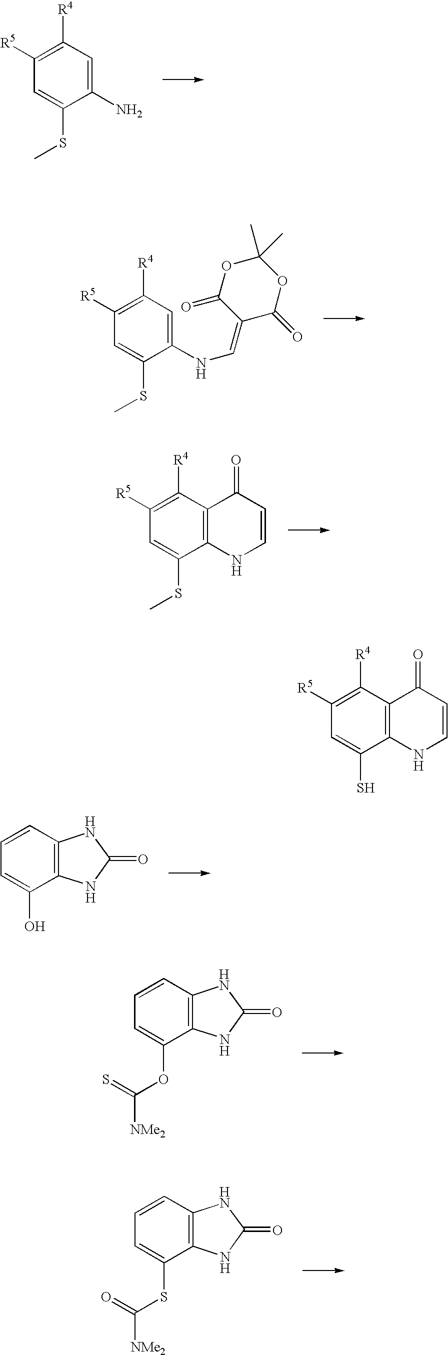

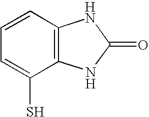

The 7-mercapto-benzimidazolone intermediate (V) can be prepared, for example according to the scheme below, by heating benzimidazolone-5-carboxylic acid with chlorosulphonic acid (Justus Liebigs Ann. Chem.; 1896 (291); 328).

The 7-chlorosulfonyl group can be reduced with a reducing agent such as triphenylphosphine to yield the 7-mercapto-benzimidazolone intermediate (IV).

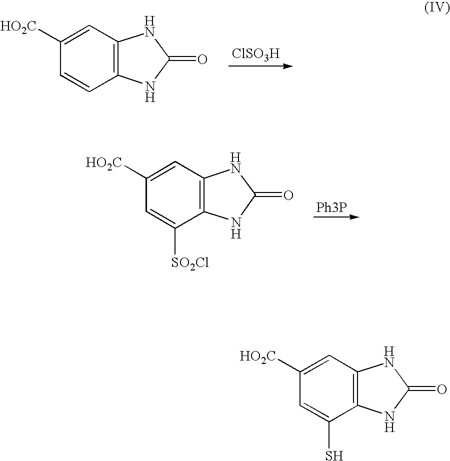

Intermediate compounds of formula (V) can be prepared using known chemistry, for example according to the scheme below: The diamino compound is reacted with carbon disulfide in an inert solvent such as dimethyl formamide (Org. Synth. 30, 1950, 56) to yield the 2-mercapto compound. Methylation of the 2-mercapto compound can be performed with iodomethane in a solvent such as acetone or methylene chloride in the presence of a base such as potassium carbonate at room temperature (J. Chem. Soc. 1949, 3311-3312, Tetrahedron, 1995, 11515-11530). Oxidation of the 2-methylthio group to the 2-methanesulfonyl group can be performed with oxidizing agents such as m-chloroperoxy benzoic acid or oxone at room temperature (J. Chem. Soc. 1949, 3311-3312, J. Heterocycl. Chem. 1995, 707-718)

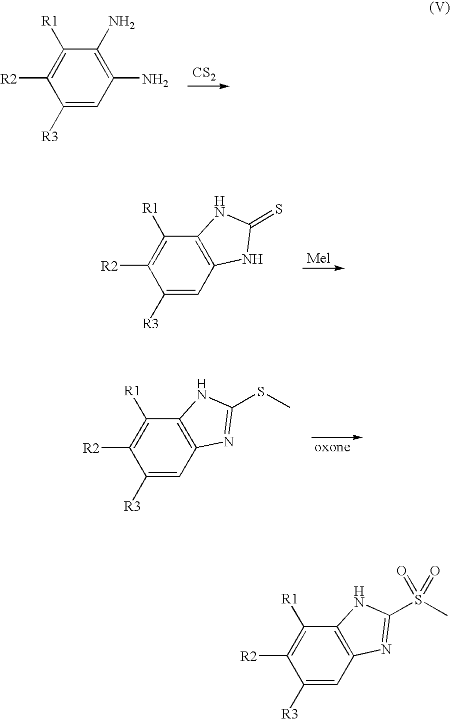

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at an appropriate stage, the addition and removal of one or more protecting groups.

The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

The compounds of formula (I) may be converted to a further compound of formula (I) using standard chemistry, for example, alkylation of amine groups. These alkylations may be performed by reacting the amine with an aldehyde in the presence of a reducing agent such as sodium triacetoxyborohydride or sodium cyanoborohydride.

The invention further provides for a process for the preparation of a compound of formula (Ia) by converting a compound of formula (VI), wherein R⁵ is carboxy and R¹, R², R³ and R⁴ are as defined in formula (I), to a compound of formula (Ia), wherein R¹, R², R³, R⁴ and R⁵ are as defined in formula (I),

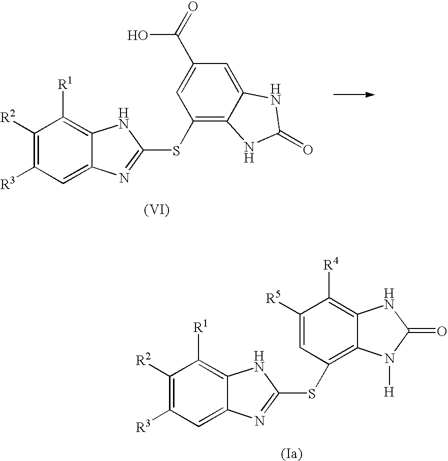

Examples of such convertions are shown in the synthetic schemes of Methods 1 to 3 below. Ester formation in method 1 can be performed in refluxing alcohol such as a lower alcohol e.g. ethanol or methanol with acid catalysis such as sulphuric acid or acidic ion exchange resin. The esters can also be synthesized from the corresponding alcohol in an inert solvent such as DMF or TBF in the presence of coupling reagents such as HATU or TBTU.

The amides in method 2 can be synthesized from the corresponding amine in an inert solvent such as DMF in the presence of coupling reagents such as 1,3-dicyclohexylcarbodiimide, 1-(3-dimethylaminopropyl)-3-ethylcarbodiimide, HATU or TBTU.

Reduction of the acid group in method 3 can be performed with a reducing agent such as diborane or by converting the acid into an ester, which is then reduced with sodium borohydride to the alcohol.
Synthetic scheme Method 1: 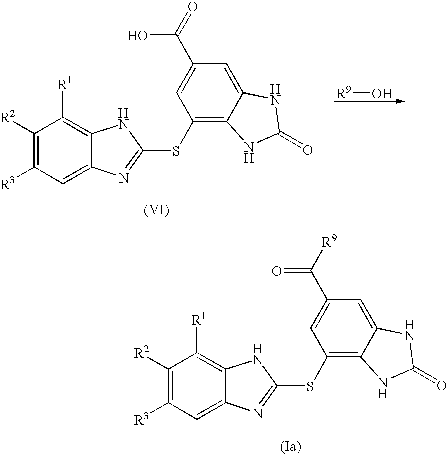
Synthetic scheme Method 2: 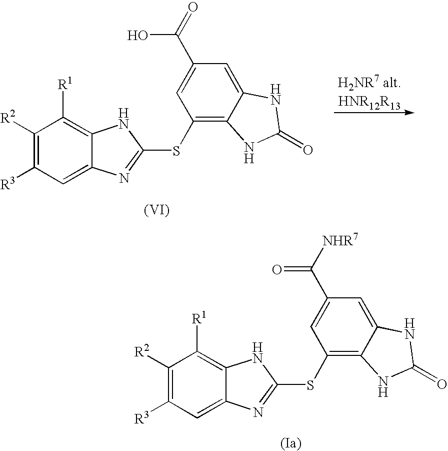
Synthetic scheme Method 3: 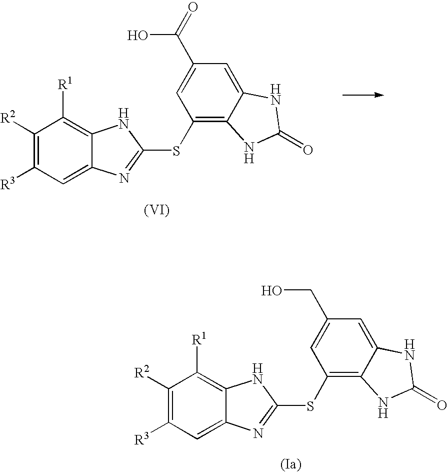
wherein R¹, R², R³, R⁷, R⁹, R¹² and R¹³ are are as defined in formula (I).

Another aspect the present invention provides an intermediate compound of formula (VI), which is used in the preparation of compounds of formula (I).

Compounds of formula (VI) may be prepared as described under processes (a) and (b).
Compounds of Formula (VI) 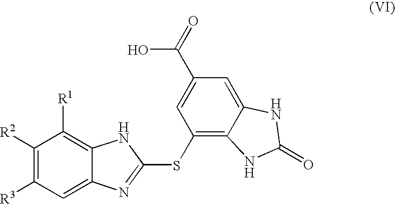
wherein:
R¹ is hydrogen, CO₂R⁷, NHCOR⁷, CN or halogen;
R² is hydrogen, halogen, OC₁₋₆ alkyl, or a 5- or 6-membered heterocyclic ring containing 1, 2 or 3 heteroatoms selected independently from N, O and S; and
R³ is hydrogen or halogen;
R⁶ is C₁₋₆ alkyl, substituted with 1, 2 or 3 substituents selected independently from hydroxy, OC₁₋₆ alkyl, OC₁₋₆ alkylOH and NR¹²R¹³;
R⁷ is CONHOC₁₋₆alkylOH, or C₁₋₆ alkyl optionally substituted with 1, 2, 3 or 4 substituents selected independently from hydroxy, OC₁₋₆ alkyl and NR¹²R¹³, or
R⁷ is phenyl or a 5- or 6-membered heterocyclic ring containing 1, 2, 3 or 4 heteroatoms selected independently from N, O and S,
   as salts, solvates or solvates of salts thereof.

The present invention farther relates to the use of compounds of formula (VI) as intermediate in the preparation of compounds of formula (I).

### EXAMPLES

The invention will now be described in more detail with the following examples that are not to be construed as limiting the invention.

All chemicals and reagents were used as received from suppliers. ¹H and ¹³C nuclear magnetic resonance (NMR) spectra were recorded on a BRUKER DPX 400 (400 MHz) spectrometer using the following solvents and references.
CDCl₃: ¹H NMR TMS (0.0 ppm) and ¹³C the central peak of CDCl₃ (77.0).
CD₃OD: ¹H NMR 3.31 ppm (central peak) and ¹³C 49.0 ppm (central peak).
DMSO-d₆: ¹H NMR 2.50 ppm (central peak) and ¹³C 39.51 ppm (central peak).
MS (ECI) were recorded on a Waters Alliance 2790+ZMD spectrometer equipped with software Mass Lynx 3.5.

Preparative HPLC were run on a Waters HPLC, column XTerra® Prep MSC₈, 10 μm, 19×300 mm, acetonitrile (20-80%)/0.1 M NH₄OAc in 1% acetonitrile, 20 ml/min. Flash column chromatography was carried out on silica gel 60 (230-400 mesh).

### EXAMPLES

### Example 1

### 8-(1H-Benzimidazol-2-ylsulfanyl)-4(1H)-quinolinone

### (i) 5-{([2-(1H-Benzimidazol-2-ylsulfanyl)anilino]methylene}-2,2-dimethyl-1,3-dioxane-4,6-dione

A solution of 2,2-dimethyl-1,3-dioxane-4,6-dione (300 mg) in trimethylorthoformate (10 ml was stirred under reflux for 1 h. 2-(1H-benzimidazol-2-ylsulfanyl)aniline (500 mg) was added and heating continued for a further 2 h. The solution was evaporated in vacuo and the residue triturated with diethyl ether to give the subtitle compound as a brown solid. (420 mg). MS: APCI(−ve) 394 (M−1).

### (ii) 8-(1H-Benzimidazol-2-ylsulfanyl)-4-(1H)-quinolinone trifluoroacetate

5-{[2-(1H-Benzimidazol-2-ylsulfanyl)anilino]methylene}-2,2-dimethyl-1,3-dioxane-4,6-dione (1.0 g) was added portionwise to diphenyl ether (20 g) at reflux and the resultant solution stirred at refluxc temperature for 15 min. The reaction was cooled to −50° C. and poured into stirring isohexane (100 ml) to give a brown precipitate which was collected by filtration and dried in vacuo. The solid was purified by reverse phase HPLC eluting with 75-05% 0.1% aqueous trifluoroacetic acid/methanol to give the title compound. (16 mg). ¹H NMR d₆-DMSO: δ 8.25 (1H, dd); 7.92 (1H, dd); 7.86 (1H, d); 7.39 (3H, m); 7.11 (2H, m); 6.13 (1H, d). M.pt. >250° C.

### Example 2

### 8-[(5-Chloro-1H-benzimidazol-2-yl)sulfanyl]-4(1H)-quinolinone

### (i) 2,2-Dimethyl-5-{[2-(methylsulfanyl)anilino]methylene}-1,3-dioxane-4,6-dione

A stirred solution of 2-(methylthio)aniline (3.74 g) and 2,2-dimethyl-5-methoxymethylene-1,3-dioxane-4,6-dione (5.0 g) in acetonitrile (40 ml) was stirred under nitrogen over night. The reaction was evaporated in vacuo and the residue triturated with diethyl ether to give the subtitle compound as a yellow solid (7.02 g). ¹H-NMR (CDCl₃): δ 11.85 (1H, d); 8.68 (1H, d); 7.53 (1H, d); 7.37 (2H, d); 7.24 (1H, m); 2.47 (3H, s); 1.77 (6H, m).

### (ii) 8-(Methylsulfanyl)-4(1H)-quinolinone

2,2-dimethyl-5-{[2-(methylsulfanyl)anilino]methylene}-1,3-dioxane-4,6-dione (7.00 g) was added portionwise to stirring diphenyl ether (60 g) at reflux over 5 min. The resultant solution was stirred under reflux for a further 15 min then cooled to ˜50° C. The reaction was poured into stirring isohexane (800 ml) to give the subtitle compound as a brown gum, which was collected and dried in vacuo (4.99 g). MS: APCI(−ve): 190 (M−1, 100%).

### (iii) Sodium 4-oxo-1,4-dihydro-8-quinoline Thiolate

A stirred solution of sodium (240 mg) in liquid ammonia (50 ml) at −33° C. was treated dropwise with a solution of 8-methylsulfanyl)-4-(1H)-quinolinone (1.0 g) in dry tetrahydrofuran (5 ml). The reaction was stirred for 30 min and quenched with ethanol (1 ml) then left to warm to room temperature. The residue was dissolved in methanol (20 ml) and evaporated in vacuo to give a brown solid, which was triturated with diethyl ether and dried in vacuo (805 Mg). ¹H-NMR (d₆-DMSO): δ 7.86 (1H, d); 7.36 (1H, dd); 7.27 (1H, dd); 6.49 (1H, t); 5.68 (1H, d).

### (iv) 8-[(5-Chloro-1H-benzimidazol-2-yl)sulfanyl]-4(1H)-quinolinone

5-Chloro-2-methanesulfonyl-1H-benzimidazole (0.3 g) (described in U.S. Pat. No. 3,480,643), sodium 4-oxo-1,4-dihydro-8-quinolinethiolate (0.35 g), acetic acid (0.3 ml) and isopropanol. (20 ml) were combined and heated under reflux conditions over night. The reaction mixture was concentrated in vacuo and water (5 ml) was added to the residue. The aqueous suspension was sonicated and insoluble material was removed by filtration. To this material was added dichloromethane (5 ml) and the sonication procedure repeated. Insoluble material was removed by filtration and treated with ethyl acetate (5 ml), sonicated and filtered again. The solid that remained was the title compound (0.05 g). MS: APCI(+ve) 328(M+1) ¹H NMR (d₆-DMSO) δ 8.29 (1H, dd); 8.02 (1H, dd); 7.83 (1H, d); 7.47-7.38 (3H, m); 7.12 (1H, dd); 6.15 (1H, d); 3.32 (2H, br. s).

### Example 3

### 2-(4-Oxo-1,4-dihydro-quinolin-8-ylsulfanyl)-1H-benzoimidazole-4-carboxylic Acid Methyl Ester

### (i) 2-Methanesulfonyl-1H-benzoimidazole-4-carboxylic Acid Methyl Ester

To a stirred solution of 2-mercapto-1H-benzoimidazole-4-carboxylic acid methyl ester (4.16 g), (ref J. Med. Cher. 1993, 36(15), 2182) in dry acetone (100 ml) was added potassium carbonate (2.76 g) and iodomethane (1.24 ml). The resulting suspension was stirred for 2 h at room temperature and was then concentrated in vacuo, water (50 ml) was added and the mixture acidified with 1 M hydrochloric acid solution. The resulting solution was washed with diethyl ether (2×50 ml) and then aqueous extract was basified with 1 M sodium hydroxide solution and extracted with dichloromethane (3×50 ml). The combined extracts were dried over anhydrous magnesium sulfate, filtered and concentrated in vacuo to 100 m. The solution was cooled in an ice bath and m-chloroperoxy benzoic acid (70% purity, 9.86 g) was added and the resulting mixture stirred at room temperature for 20 h. The reaction mixture was washed with saturated sodium hydrogen carbonate solution (2×50 ml), dried over anhydrous magnesium sulfate, filtered and concentrated in vacuo to afford the sub-title compound. Yield 4.52 g. MS: APCI(+ve) 255 (M+1) ¹H NMR (CDCl₃) δ11.09 (1H, s); 8.12 (2H, t); 7.49 (1H, t); 4.04 (3H, s); 4.04 (3H,s).

### (ii) 2-(4-Oxo-1,4-dihydro-quinolin-8-ylsulfanyl)-1H-benzoimidazole-4-carboxylic Acid Methyl Ester

To a stirred solution of 2-methanesulfonyl-1H-benzoimidazole-4-carboxylic acid methyl ester (1.02 g) in dry isopropanol (50 ml) was added 8-mercapto-1H-quinolin-4-one sodium salt (1.07 g) and acetic acid (0.5 ml). The resulting suspension was stirred at 90° C. for 48 h, cooled and concentrated in vacuo. The residue was partitioned between ethyl acetate (50 ml) and water (20 ml) and was extracted into 1 M hydrochloric acid solution (2×30 ml). The aqueous extracts were neutralised with 1 M sodium hydroxide solution and were extracted into ethyl acetate (3×30 ml), dried over anhydrous magnesium sulfate, filtered and concentrated in vacuo. The resulting solid was triturated with acetone and collected by filtration to afford the title compound as a solid. Yield 0.01 g. MS: APCI(+ve) 352 (M+1) ¹H NMR (399.98 MHz, DMSO) δ 12.86 (1H,s); 11.26 (1H, d); 8.29 (1H, dd); 8.05 (1H, dd); 7.79-7.73 (2H, m); 7.66 (1H, d); 7.41 (1H, t); 7.18 (1H, t); 6.12 (1H, d); 3.97 (3H, s). MP: >250° C.

### Example 4

### 8-[(5-Chloro-1H-benzimidazol-2-yl)sulfanyl]-5-nitro-4(1H)-quinolinone

### (i) 5-[(2-fluoro-5-nitroanilino)methylene]-2,2-dimethyl-1,3-dioxane-4,6-dione

A solution of 2-fluoro-5-nitroaniline (25.0 g) in acetonitrile (300 ml) was treated with 5-(methoxymethylene)-2,2-diethyl-1,3-dioxane-4,6-dione (29.75 g) and stirred at room temperature for 20 h. The solvent was removed in vacuo and the residue triturated with 20% diethyl ether in isohexane (300 ml). The resultant pale brown solid was collected by filtration and dried in vacuo. Yield 49.08 g. ¹H NMR CDCl₃: δ 11.47 (1H, d); 8.72 (1H, d); 8.36-8.34 (1H, m); 8.17-8.13 (1H, m); 7.41 (1H, t); 1.78 (6H, s)

### (ii) 8-Fluoro-5-nitro-4(1H)quinolinone

5-[(2-fluoro-5-nitroanilino)methylene]-2,2-dimethyl-1,3-dioxane-4,6-dione was added cautiously portionwise to stirred diphenyl ether (600 ml) at reflux (CO₂ evolved). After addition was complete, heating was continued for 10 min and the reaction left to cool to ˜50° C. The dark solution was poured slowly into stirred isohexane (4 l) and the resultant precipitate collected by filtration. The solid was triturated repeatedly with dichloromethane (5×200 ml) to give a dark brown solid, which was dried in vacuo. Yield 20.21 g. MS: APCI(+ve) 209 (M+1).

### (iii) 8-[(5-Chloro-1H-benzimidazol-2-yl)sulfanyl]-5-nitro-4(1H)-quinolinone

A stirred suspension of 5-chloro-1H-benzimidazole-2-thiol (46 mg) in ethanol (2 ml) was treated with 10 M sodium hydroxide solution (25 μl) and stirred for 10 min to give a dark solution. 8-Fluoro-5-nitro-4(1H)-quinolinone (54 mg) was added and the reaction was stirred at 100° C. over night. The solvent was evaporated in vacuo and the residue purified by reverse phase HPLC eluting with 75-05% 0.1% aqueous trifluoroacetic acid/methanol to give the title compound. Yield 13 mg. ¹H NMR d₆-DMSO: δ 8.15 (1H, d); 7.92 (1H, d); 7.64 (1H, d); 7.51 (1H, d); 7.41 (1H, d); 7.16 (1H, dd); 6.23 (1H, d).

### Example 5

### 5-Amino-8-[(5-chloro-1H-benzimidazol-2-yl)sulfanyl]-4(1H)-quinolinone

A stirred suspension of 8-[(5-chloro-1H-benzimidazol-2-yl)sulfanyl]-5-nitro-4(1)-quinolinone (5.30 g) in acetic acid (200 ml) was hydrogenated over 10% palladium on charcoal (500 mg) at 5 bar for 8 days. The suspension was filtered through celite and the celite pad washed with acetic acid (5×200 ml). The filtrate was evaporated in vacuo (azeotroped twice with toluene) and the residue triturated with diethyl ether (200 ml) to give the crude amine (3.20 g). A sample of the crude amine (500 mg) was triturated with methanol (2×5 ml) and diethyl ether (5 ml) then dried in vacuo. Yield 305 mg. ¹H NMR d₆-DMSO: δ12.16 (1H, s); 10.96 (1H, s); 7.59-7.50 (3H, m); 7.31-7.28 (1H, s); 7.10 (1H, d), 6.41 (1H, d); 5.94 (1H, d). M.pt. >280° C.

### Example 6

### 8-[(5-Chloro-1H-benzimidazol-2-yl)sulfanyl]-5-{[(2R)-pyrroldinylmethyl]amino}-4(1H)-quinolinone

### (i) Tert-butyl (2R)-2-[({8-[(5-chloro-1H-benzimidazol-2-yl)sulfanyl]-4-oxo-1,4-dihydro-5-quinolinyl}amino)methyl]-1-pyrrolidinecarboxylate

A stirred solution of N-boc-D-prolinal (233 mg) in acetic acid (5 ml) was treated with 5-amino-8-[(5-chloro-1H-benzimidazol-2-yl)sulfanyl]-4(1H)-quinolinone (200 mg) and stirred for 1 h. Sodium triacetoxyborohydride (375 mg) was added and stirring was continued over night. The mixture was evaporated in vacuo to give a dark solid, which was used without purification. Yield 320 mg. MS: APCI(+ve) 526 (M+1).

### (ii) 8-[(5-Chloro-1H-benzimidazol-2-yl)sulfanyl]-5-{[(2R)-pyrrolidinylmethyl]amino}-4(1H)-quinolinone

A stirred suspension of tert-butyl (2R)-2-[({8-[(5-chloro-1H-benzimidazol-2-yl)sulfanyl]-4-oxo-1,4-dihydro-5-quinolinyl}amino)methyl]-1-pyrrolidinecarboxylate (320 mg) in dichloromethane (20 ml) was treated with trifluoroacetic acid (5 ml) and stirred at room temperature for 1 h. The solution was evaporated in vacuo and the residue purified by reverse phase HPLC eluting with 75-05% 0.1% aqueous trifluoroacetic acid/methanol to give the title compound as a yellow solid. Yield 48 mg. ¹H NMR d₆-DMSO: δ10.89 (1H, br s); 9.03 (1H, br s); 8.67 (1H, br s); 7.72 (1H, d); 7.64 (1H, d); 7.43 (1H, d); 7.38 (1H, d); 7.13 (1H, m); 6.53 (1H, d); 6.05 (1H, d); 3.80 (1H, m); 3.57 (2H, m); 3.22 (2H, m); 2.15 (1H, m); 1.93 (2H, m); 1.68 (1H, m).

### Example 7

### 8-[(5-Chloro-1H-benzimidazol-2-yl)sulfanyl]-5-[(4-piperidinylmethyl)amino]-4(1H)-quinolinone

A solution of 5-amino-8-[(5-chloro-1H-benzimidazol-2-yl)sulfanyl]-4(1H)-quinolinone (300 mg) and tert-butyl 4-formyl-1-piperidinecarboxylate (373 mg) in acetic acid was stirred at room temperature for 30 min. Sodium triacetoxyborohydride (560 mg) was added and stirring continued for 2 h. The mixture was evaporated in vacuo and the residue dissolved in dichloromethane (20 ml) and treated with trifluoroacetic acid (10 ml). After 1 h the reaction was evaporated in vacuo and the residue dissolved in methanol (5 ml) and loaded onto a cartridge of SCX silica (5 g). Impurities were eluted from the column with methanol (4×15 ml) and the product eluted with 10% aqueous ammonia (S.G. 0.88) in methanol (5×20 ml). The product eluent was evaporated in vacuo and the residue purified by reverse phase HPLC eluting with 50-05% 0.1% aqueous ammonia (S.G. 0.88)/methanol to give the title compound as a green solid (34 mg). ¹H NMR d₆-DMSO: δ 10.77 (1H, br s); 7.62 (2H, dd); 7.40 (1H, d); 7.34 (1H, d); 7.06 (1H, dd); 6.36 (1H, d); 5.99 (1H, d); 3.09 (2H, m); 2.97 (2H, m); 2.54 (2H, m); 1.72 (2H, m); 1.18 (2H, m). M.Pt 230-232° C.

### Example 8

### 5-Chloro-2-[(4-oxo-1,4-dihydroquinolin-8-yl)thio]-1H-benzimidazole-4-carbonitrile

### (i) 2-Amino-6-chloro-3-nitrobenzonitrile

2,6-Dichloro-3-nitrobenzonitrile (100 g) was suspended in methanol (1.2 l) and the mixture saturated with gaseous ammonia. After 4 days the mixture was filtered to give 2-amino-6-chloro-3-nitrobenzonitrile as a bright yellow solid (60 g). ¹H NMR d₆-DMSO: δ 8.32 (1H, dd), 7.82 (2H, broad s), 6.96 (1H, dd).

### (ii) 2,3-Diamino-6-chlorobenzonitrile

2-Amino-6-chloro-3-nitrobenzonitrile (8 g) in acetic acid (100 ml) was treated with 5% palladium on carbon (1 g) and the mixture hydrogenated at 20 atmospheres for 16 h. The catalyst was filtered off and the acetic acid removed in vacuo to give 2,3-diamino-6-chlorobenzonitrile. ¹H NMR d₆-DMSO: δ 6.63(1H,dd), 6.55 (1H, dd), 5.73 (2H, broad s), 5.13 (2H, broad s).

### (iii) 5-Chloro-2-mercapto-1H-benzimidazole-4-carbonitrile

2,3-Diamino-6-chlorobenzonitrile (6 g) was treated with a 50% solution of carbon disulfide in dimethyl formamide (100 ml) and the solution heated in a sealed bomb at 70° C. for 16 h. After cooling the bomb was vented and the mixture poured into water (500 ml). The product was collected by filtration (8 g). ¹H NMR d₆-DMSO: δ 13.71 (1H, broad s), 13.11 (1H, broad s), 7.37 (2H, S).

### (iv) 5-Chloro-2-(methylthio)-1H-benzimidazole-4-carbonitrile

5-Chloro-2-mercapto-1H-benzimidazole-4-carbonitrile (8 g) in acetone (150 ml was treated with solid potassium carbonate (8 g). The suspension was carefully titrated at room temperature with methyl iodide (0.5 g aliquots) using IC-MS to ascertain disappearance of starting material and appearance of S-methylated material. When all the starting material had been consumed, the acetone was removed in vacuo and the residue partitioned between water and ethyl acetate to give a brown solid (8 g). ¹H NMR d₆-DMSO at 90° C.: δ 12.81 (1H, broad s), 7.86 (1H, broad s), 7.4 (2H, broad m), 2.72 (3H, broad s).

### (v) 5-chloro-2-(methylsulfonyl)-1H-benzimidazole-4-carbonitrile

5-Chloro-2-(methylthio)-1H-benzimidazole-4-carbonitrile (1 g) in methanol (10 ml)was treated with oxone (4.68 g) in water (10 ml) and stirred over night. The methanol was removed in vacuo and the reaction mixture treated with aqueous sodium bicarbonate. The required product was extracted with ethyl acetate, dried and the solvent removed in vacuo to give 5-chloro-2-(methylsulfonyl)-1H-benzimidazole-4-carbonitrile (1 g). ¹H NMR d₆-DMSO: δ 8.17 (1H, d), 7.71 (1H, dd), 3.58 (3H, s).

### (vi) 5-Chloro-2-[(4-oxo-1,4-dihydroquinolin-8-yl)thio]-1H-benzimidazole-4-carbonitrile

5-Chloro-2-(methylsulfonyl)-1H-benzimidazole-4-carbonitrile (0.2 g) and sodium 4-oxo-1,4-dihydro-8-quinolinethiolate (0.21 g) were added to isopropanol (10 ml) and glacial acetic acid (0.2 ml) then heated in a sealed tube at 80° C. over night. After cooling to room temperature, insoluble material was removed from the reaction mixture and this was washed sequentially with water, methanol and dichloromethane. The remaining beige solid was the desired product (0.05 g, 18%). ¹H NMR d₆-DMSO: δ 12.87 (1H, br s); 11.40 (1H, br s); 8.35 (1H, d); 8.12 (1H, d); 7.79 (1H, t); 7.62 (1H, d); 7.46 (1H, t); 7.39 (1H, d); 6.14 (1H, d). MS: APCI (+ve) 353 (M+1). M.pt. 376° C. (decomposes).

### Example 9

### Methyl 5-chloro-2-[(4-oxo-1,4-dihydroquinolin-8-yl)thio]-1H-benzimidazole-4-carboxylate

### (i) 5-Chloro-2-mercapto-1H-benzimidazole-4-carboxylic Acid

5-Chloro-2-mercapto-1H-benzimidazole-4-carbonitrile (0.5 g) was added to ethanol (5 ml) and 10 M sodium hydroxide solution (5 ml). The mixture was heated, under reflux conditions, for 24 h then diluted with water (10 ml) and washed with ethyl acetate (25 ml). The aqueous layer was acidified by addition of 2 M hydrochloric acid and product was extracted with ethyl acetate. The ethyl acetate was washed with brine (20 ml), dried over anhydrous magnesium sulfate, filtered and concentrated in vacuo to give a pale brown solid, 0.26 g (50%). ¹H NMR d₆-DMSO: δ 12.87 (1H, br s); 12.52 (1H, br s); 7.28-7.19 (2H, m); 3.33 (1H, br s).

### (ii) Methyl 5-chloro-2-(methylthio)-1H-benzimidazole-4-carboxylate

5-Chloro-2-mercapto-1i-benzimidazole-4-carboxylic acid (0.26 g), potassium carbonate (0.62 g) and acetone (10 ml) were combined and treated with iodomethane (0.28 ml). The reaction mixture was stirred at room temperature for 1 h, after which time the thiol had been methylated. The reaction mixture was concentrated in vacuo and methanol (3 ml) was added to the residue. The methanolic suspension was passed through an acrodisc™ to remove potassium carbonate and the filtrate was treated with concentrated sulfuric acid (4 drops). This was heated under reflux for 3 days then concentrated in vacuo. The residue was partitioned between ethyl acetate and saturated sodium bicarbonate solution. The ethyl acetate was washed with brine, dried over anhydrous magnesium sulfate, filtered and concentrated in vacuo to give an oil (0.16 g, 62%). ¹H NMR d₆-DMSO: δ 12.95 and 12.62 (1H, 2 br s); 7.68 and 7.49 (1H, 2 br d); 7.27 (1H, d); 3.93 (3H, s); 2.69 (3H, s).

### (iii) Methyl 5-chloro-2-(methylsulfonyl)-1H-benzimidazole-4-carboxylate

Methyl 5-chloro-2-(methylthio)-1H-benzimidazole-4-carboxylate (0.16 g) was added to methanol (10 ml) and the resulting suspension was treated with an solution of oxone™ (0.61 g) in water (10 ml). The mixture was stirred at room temperature for 1 h then the methanol was removed by evaporation in vacuo. The remaining aqueous suspension was neutralised by addition of saturated sodium bicarbonate solution and product was extracted with ethyl acetate. The ethyl acetate was washed with brine, dried over anhydrous magnesium sulfate, filtered and concentrated in vacuo to give a solid, which was used without purification in the next step, (0.15 g, 87%).

### (iv) Methyl 5-chloro-2-[(4-oxo-1,4-dihydroquinolin-8-yl)thio]-1H-benzimidazole-4-carboxylate

The titled compound was prepared from methyl 5-chloro-2-(methylsulfonyl)-1H-benzimidazole-4-carboxylate using the method described for Example 8 (vi). ¹H NMR d₆-DMSO: δ 8.29 (1H, d); 8.03 (1H, d); 7.81 (1H, d); 7.49 (1H, d); 7.41 (1H, t); 7.19 (1H, d); 6.14 (1H, d); 3.93 (3H, s).

### Example 10

### N-{5-Chloro-2-[(4-oxo-1,4-dihydroquinolin-8-yl)thio]-1H-benzimidazol-4-yl}benzamide

### (i) 5-Chloro-4-nitro-1H-benzimidazole-2-thiol

A stirred solution of 4-chloro-3-nitrobenzene-1,2-diamine (9.20 g) in dry dimethylformamide (30 ml) was treated with carbon disulfide (25 ml) and stirred at room temperature for 72 h. The reaction was poured into stirring, distilled water (500 ml) and the resultant suspension degassed with nitrogen. The mixture was filtered and the solid washed with distilled water (2×50 ml) to give a brown solid, which was dried in vacuo. Yield 8.44 g. ¹H NMR d₆-DMSO: δ 13.23 (2H, br s), 7.42 (1H, d), 7.35 (1H, d).

### (ii) 5-Chloro-2-(methylthio)-4-nitro-1H-benzimidazole

A stirred suspension of 5-chloro-4-nitro-1H-benzimidazole-2-thiol (8.80 g) and potassium carbonate (5.29 g) in acetone (100 ml) was treated with methyl iodide (2.37 ml) and stirred at room temperature for 2 h. The mixture was evaporated in vacuo and the residue treated with distilled water (200 ml). The mixture was extracted with ethyl acetate (2×200 ml) and the organics dried over anhydrous magnesium sulfate and evaporated in vacuo to give a brown solid. Yield 8.91 g. ¹H NMR d₆-DMSO: δ 13.40 (1H, br s), 7.64 (1H, d), 7.40 (1H, d), 2.71 (3H, s).

### (iv) 5-Chloro-2-(methylthio)-1H-benzimidazol-4-amine

A suspension of 5-chloro-2-(methylthio)-4-nitro-1H-benzimidazole (5.00 g) and iron powder (8.05 g) in a mixture of ethanol (105 ml), water (33 ml), acetic acid (11 ml) and formic acid (2 ml) was stirred under reflux conditions for 2 h. The mixture was filtered and the filtrate evaporated in vacuo. The residue was treated with saturated sodium bicarbonate solution (400 ml) and extracted with ethyl acetate (2×300 ml). The extracts were dried over anhydrous magnesium sulfate and evaporated in vacuo to give a dark solid. Yield 3.71 g. ¹H NMR d₆-DMSO: δ 12.41 (1H, br s), 6.95 (1H, d), 6.61 (1H, d), 5.23 (2H br s), 2.68 (3H, s).

### (v) N-[5-Chloro-2-(methylthio)-1H-benzimidazol-4-yl]benzamide

A stirred solution of 5-chloro-2-(methylthio)-1H-benzimidazol-4-amine (800 mg) in dichloromethane (20 ml) was treated with benzoyl chloride (1.3 ml) and triethylamine (1.57 ml). The resultant suspension was stirred at room temperature over night and evaporated in vacuo. The residue was suspended in methanol:water (3:1, 50 ml) and stirred at 60° C. over night. The reaction was evaporated in vacuo and the residue partitioned between ethyl acetate (100 ml) and water (100 ml). The organic layer was dried over anhydrous magnesium sulfate, evaporated in vacuo and the residue purified by flash column chromatography eluting with 20% ethyl acetate in isohexane. The subtitle product was obtained as an orange solid. Yield 430 mg. ¹H NMR d₆-DMSO: δ 12.69 (1H, br s), 10.25 (1H, br s), 8.07 (2H, m), 7.65-7.52 (3H, m), 7.47 (1H, d), 7.25 (1H, d), 2.68 (3H, s).

### (vi) N-[5-Chloro-2-(methylsulfonyl)-1H-benzimidazol-4-yl]benzamide

A stirred solution of N-[5-chloro-2-(methylthio)-1H-benzimidazol-4-yl]benzamide (430 mg) in methanol (25 ml) was treated with a solution of oxone (1.33 g) in water (10 ml) and stirred at room temperature for 24 h. The mixture was diluted with methanol (50 ml) and filtered. The filtrate was evaporated in vacuo to give an off-white solid. Yield 300 mg. ¹H NMR d₆-DMSO: δ10.41 (1H, br s), 8.09 (2H, m), 7.74 (1H, d), 7.64 (1H, m), 7.59-7.53 (3H, m), 3.52 (3H, s).

### (vii) N-{5-Chloro-2-[(4-oxo-1,4-dihydroquinolin-8-yl)thio]-1H-benzimidazol-4-yl}benzamide

A suspension of N-[5-chloro-2-(methylsulfonyl)-1i-benzimidazol-4-yl]benzamide (100 mg) and sodium 4-oxo-1,4-dihydro-8-quinoline thiolate (51 mg) in isopropanol (10 ml) was treated with acetic acid (2 drops) and stirred at 80° C. over night. The reaction was evaporated in vacuo and the residue purified by flash column chromatography eluting with 2% methanol in dichloromethane. The title compound was obtained as a yellow solid. Yield 18 mg. M.pt 280-285° C., ¹H NMR d₆-DMSO: δ 13.05 (1H, br s), 11.35 (1H, br s), 10.25 (1H, br s), 8.28 (1H, d), 8.05 (2H, br d), 7.81 (1H, s), 7.58 (2H, m), 7.45-7.15 (4H, m), 6.97-6.80 (1H, m), 6.13 (1H, m).

### Example 11

### N{5-Chloro-2-[(4-oxo-1,4-dihydroquinolin-8-yl)thio]-1H-benzimidazol-4-yl}-2-furamide

### (i) N-[5-chloro-2-(methylthio)-1H-benzimidazol-4-yl]-2-furamide

The subtitle compound was prepared from 5-chloro-2-(methylthio)-1H-benzimidazol-4 amine (800 mg) and 2-furoyl chloride (1.1 ml) according to the method of Example 10 (v). Yield 300 mg. ¹H NMR d₆-DMSO: δ 12.69 (1H, br s), 10.15 (1H, br s), 7.95 (1H, d), 7.47 (1H, d), 7.35 (1H, m), 7.24 (1H, dd), 6.72 (1H, m), 2.68 (3H, s).

### (ii) N-[5-chloro-2-(methylsulfonyl)-1H-benzimidazol-4-yl]-2-furamide

The subtitle compound was prepared from N-[5-chloro-2-(methylthio)-1H-benzimidazol-4-yl]-2-furamide (300 mg) according to the method of Example 10 (vi). Yield 280 mg. ¹H NMR d₆-DMSO: δ 10.34 (1H, s), 7.98 (1H, m), 7.72 (1H, d), 7.53 (1H, d), 7.37 (1H, dd), 6.74 (1H, dd), 3.17 (3H, s).

### (iii) N-{5-chloro-2-[(4-oxo-1,4 dihydroquinolin-8-yl)thio-]-1H-benzimidazol-4-yl)-2-furamide

The title compound was prepared from N-[5-chloro-2-(methylsulfonyl)-1H-benzimidazol-4-yl]-2-furamide) (100 mg) according to the method of example 10 (vii). Yield 13 mg. ¹H NMR 6-DMSO: δ 13.05 (1H, br s), 11.35 (1H, br s), 10.20 (1H, br s), 8.28 (1H, d), 8.00 (2H, br m), 7.81 (1H, m), 7.38 (3H, m), 7.22 (1H, m), 6.74 (1H, m), 6.13 (1H, d).

### Example 12

### 7-[(5-Chloro-1H-benzimidazol-2-yl)thio]-3-(methylsulfonyl)-1H-indol-4-amine

### (i) 7-Fluoro-3-(methylthio)-4-nitro-1,3-dihydro-2H-indol-2-one

A stirred solution of ethyl (methylthio)acetate (28.5 ml) in dichloromethane (600 ml) at −70° C. was treated dropwise with sulfuryl chloride (16.5 ml) and stirred for 30 min. A solution of 2-fluoro-5-nitroaniline (28 g) and N,N,N′,N′-tetramethylnaphthalene-1,8-diamine (46.2 g) in dichloromethane (450 ml) was added dropwise at −70° C. and the resultant red mixture stirred for 2 h. Triethylamine (29.8 ml) was added dropwise and stirring continued at −70° C. for a further 1 h. The cooling bath was removed and the reaction stirred to room temperature over night then diluted with dichloromethane (500 ml) and washed with saturated brine (3×500 ml). The organics were dried over anhydrous magnesium sulfate and evaporated in vacuo to give a red oil which was dissolved in glacial acetic acid (500 ml) and stirred at room temperature for 1 h. The solution was evaporated in vacuo and the residue triturated with 2 M hydrochloric acid (400 ml). The solid was collected by filtration, triturated with diethyl ether (2×100 ml) and dried in vacuo. Yield 30.6 g. ¹H NMR d₆-DMSO: δ 11.51 (1H, s), 7.81 (1H, dd), 7.48 (1H, t), 1.92 (3H, s).

### (ii) 7-Fluoro-3-(methylthio)+nitro-1H-indole

A solution of 7-fluoro-3-(methylthio)-4-nitro-, 1,3-dihydro-2H-indol-2-one (0.2 g) in dry tetrahydrofuran (10 ml) was treated with 1 M borane in tetrahydrofuran (1.66 ml). The reaction mixture was stirred at room temperature, under nitrogen, for 20 h. A further (1.66 ml) of 1 M borane in tetrahydrofuran was then added dropwise to the reaction mixture and stirring was continued for a further one h. The reaction mixture was quenched with a mixture of 2 M hydrochloric acid and methanol and then basified with saturated sodium hydrogen carbonate solution. The product was extracted into ethyl acetate (×3) and the combined extracts were dried over anhydrous magnesium sulfate, filtered and then concentrated in vacuo. The resulting solid was purified by flash chromatography using 10%-20% ethyl acetate in iso-hexane as eluent. The sub-title compound was obtained as a bright red solid. Yield 81.6 mg. ¹H NMR d₆-DMSO: δ 7.808-7.729 (1H, dd): 7.729 (1H, s): 7.201-7.139 (1H, t): 2.373 (3H, s). MS: APCI (−ve) 225(M−H).

### (iii) 7-Fluoro-3-(methylsulfonyl)-4-nitro-1H-indole

A solution of 7-fluoro-3-(methylthio)-4-nitro-1H-indole (0.463 g) in methanol was treated with a solution of oxone (1.6 eq, 2.02 g) in water and the resulting suspension was stirred at room temperature for 30 h. A further 1 eq of oxone in water was added to the reaction mixture which was then stirred at room temperature for a further 24 h. The methanol was removed in vacuo and the residue was neutralized with aqueous sodium hydrogen carbonate. The product was extracted into ethyl acetate (×3) and the combined extracts were washed with saturated sodium metabisulfite solution and brine and then dried over anhydrous magnesium sulfate. After filtration the solvent was removed in vacuo to give the sub-title compound as a yellow solid. Yield 439 mg. ¹H NMR 300 MHz d₆-DMSO: δ 8.363 (1H, s); 7.923-7.880 (1H, dd); 7.394-7.332 (1H, t); 3.410 (3H, s).

### (iv) 5-Chloro-2-{[3-(methylsulfonyl)-4-nitro-1H-indol-7-yl]thio}-1H-benzimidazole

A solution of 5-chlorobenzimidazole-2-thiol (0,356 g) in N-propanol was treated with potassium hydroxide (1.67 M, 1.16 ml). The resulting suspension was heated at reflux temperature for 0.5 h and then allowed to cool. 7-Fluoro-3-(methylsulfonyl)-4-nitro-1H-indole was added and the resulting mixture heated under reflux conditions for 20 h. After cooling, the reaction mixture was quenched with water and the product was then extracted into ethyl acetate (×3). The combined organic layers were washed with 2 M sodium hydroxide solution (×7), dried over anhydrous magnesium sulfate, filtered and evaporated in vacuo. The crude residue was purified using flash chromatography eluting with 20% ethyl acetate in dichloromethane to give the sub-title compound as a yellow solid. Yield 200 mg. ¹H NMR 300 MHz d₆-DMS: δ 8.810 (1H, s); 8.042 (1H, s); 7.893-7.856 (1H, d ); 7.772-7.744 (1H, d); 7.651-7.646 (1H, d); 7.575-7.546 (1H,d); 7.321 (1H, br d); 7.233-7.205 (2H, d); 3.503 (3H, s).

### (v) 7-[(5-Chloro-1H-benzimidazol-2-yl)thio]-3-(methylsulfonyl)-1H-indol-4-amine

A suspension of 5-chloro-2-{[3-(methylsulfonyl)-4-nitro-1H-indol-7-yl]thio}-1H-benzimidazole(100 mg) and tin (II) chloride(224 mg) in ethanol was heated to reflux temperature for 7 h. The reaction mixture was allowed to cool and half of the ethanol was removed in vacuo. The residue was poured into water and basified by the addition of saturated sodium bicarbonate solution. The resulting mixture was filtered through celite and the product was extracted into ethyl acetate (×3). The combined organic extracts were washed with brine, dried over anhydrous magnesium sulfate, filtered and then concentrated in vacuo. The crude residue was purified using reverse phase HPLC eluting with 75%-05% 0.1% TFA/acetonitrile to give the title compound as a brown solid. Yield 14.4 mg. ¹H NMR 400 MHz d₆-DMSO: δ 7.703 (1H, s); 7.382-7.378 (1H, s); 7.350-7.306 (2H, dd); 7.083-7.056 (1H, d); 6.562-6.542 (1H, d); 3.254-3.229 (3H, s). MS: APCI(+ve) 393 (M+1).

### Example 13

### Methyl 5-chloro-2-{[3-(methylsulfonyl)-1H-indol-7-yl]thio}-1H-benzimidazole-4-carboxylate

### (i) 3-(Methylsulfonyl)-7-(methylthio)-4-nitro-1H-indole

A solution of 7-fluoro-3-(methylsulfonyl)-4-nitro-1H-indole (2.00 g) and sodium thiomethoxide (800 mg) in dry dimethylformamide (10 ml) was stirred at 100° C. for 2 h. The mixture was poured into saturated brine (200 ml) and extracted with ethyl acetate (3×100 ml). The extracts were washed with saturated brine (3×200 ml), dried over anhydrous magnesium sulfate and evaporated in vacuo to give a yellow solid. Yield 2.06 g. ¹H NMR d₆-DMSO: δ 13.04 (1H, s), 8.19 (1H, d), 7.88 (1H, d), 7.33 (1H, d), 3.42 (3H, s), 2.71 (3H, s).

### (ii) 3-(Methylsulfonyl)-7-(methylthio)-1H-indol-4-amine

A suspension of 3-(methylsulfonyl)-7-(methylthio)-4-nitro-1H-indole (2.06 g) and iron powder (2.82 g) in ethanol:water acetic acid:formic acid (70:22:7:1, 100 ml) was stirred under reflux for 2 h. The mixture was cooled to room temperature and filtered. The filtrate was evaporated in vacuo and the residue triturated with saturated sodium bicarbonate solution (50 ml). The resultant solid was collected by filtration and dried in vacuo at 70° C. Yield 2.00 g. ¹H NMR d₆-DMSO: δ 13.09 (1H, br s), 12.03 (1H, br s), 7.73 (1H, s), 7.15 (1H, d), 6.44 (1H, d), 5.79 (1H, br s), 3.26 (3H, s), 2.33 (3H, s).

### (iii) 3-(Methylsulfonyl)-7-(methylthio)-1H-indole

A stirred solution of 3-methylsulfonyl)-7-(methylthio)1H-indol-4-amine (2.0 g) in 25% aqueous hypophosphorous acid (100 ml) at 0° C. Was treated dropwise with a solution of sodium nitrite (1.08 g) in water (15 ml). The resultant mixture was stirred at 0° C. for 1 h and then at room temperature for 3 h. The reaction was poured into brine (500 ml) and extracted with ethyl acetate (3×300 ml). The extracts were dried over anhydrous magnesium sulfate and evaporated in vacuo to give a dark solid, which was purified by flash column chromatography eluting with 25% ethyl acetate in isohexane. The subtitle product was obtained as a pale yellow solid. Yield 730 mg. ¹HNMR d₆-DMSO: δ 12.28 (1H, br s), 7.94 (1H d), 7.68 (1H, dd), 7.27 (2H, m), 3.19 (3H, s), 2.56 (3H, s).

### (iv) Sodium 3-(methylsulfonyl)-1H-indole-7-thiolate

A stirred solution 3-(methylsulfonyl)-7-(methylthio)-1H-indole (200 mg) in liquid ammonia (10 ml) was treated portionwise with sodium (38 mg). The cooling bath was removed and the solvent allowed evaporating over 1 h. The residue was quenched with ethanol (10 ml) and evaporated in vacuo to give a brown solid. (180 mg). MS: APCI(−ve) 226 (M−1).

### (v) Methyl 5-chloro-2-{[3-(methylsulfonyl)-1H-indol-7-yl]thio}-1H-benzimidazole-4-carboxylate

A stirred solution of sodium 3-(methylsulfonyl)-1H-indole-7-thiolate (180 mg) and methyl 5-chloro-2-(methylsulfonyl)-1i-benzimidazole-4-carboxylate (191 mg) in isopropanol (10 ml) was treated with glacial acetic acid (2 drops) and stirred at 100° C. in a sealed tube over night. The reaction was evaporated in vacuo and the residue purified by flash column chromatography eluting with 30% ethyl acetate in isohexane to give the title product as an off-white solid. Yield 28 mg. M.pt. 284-286° C. ¹H NMR d₆-DMSO: δ 12.87 (1H, br s), 12.35 (1H, br s), 8.00 (2H, m), 7.61 (1H, d), 7.55 (1H, d), 7.35 (1H, t), 7.22 (1H, d), 3.95 (3H, d), 3.25 (3H, s).

### Example 14

### 5-Chloro-2-[(3-methyl-4-nitro-1H-indol-7-yl)thio]-1H-benzimidazole

### (i) 7-Fluoro-3-methyl-3-(methylthio)-4-nitro-1,3-dihydro-2H-indol-2-one

A stirred suspension of 7-fluoro-3-(methylthio)-1-nitro-1,3-dihydro-2H-indol-2-one (5.0 g) and sodium carbonate (36 g) in acetone: dimethylformamide (2:1, 200 ml) was treated with methyl iodide (1.3 ml) and stirred at room temperature for 3 h. The reaction was evaporated in vacuo and the residue treated with distilled water (600 ml) and extracted with ethyl acetate (3×300 ml). The organics were washed with saturated brine (2×250 ml), dried over anhydrous magnesium sulfate and evaporated in vacuo to give a red solid which was purified by flash column chromatography eluting with 0-1% methanol in dichloromethane. Yield 1.15 g. ¹H NMR d₆-DMSO: δ 11.70 (1H, br s), 7.79 (1H, dd), 7.51 (1H, t), 1.76 (3H, s), 1.75 (3H, s).

### (ii) 7-Fluoro-3-methyl-4-nitro-1H-indole

A solution of 7-fluoro-3-methyl-3-(methylthio)+nitro-1,3-dihydro-2H-indol-2-one (800 mg) in dry tetrahydrofuran (15 ml) under nitrogen was treated with borane dimethyl sulfide complex (1.48 ml) and stirred under reflux for 2 h. The reaction was poured into 1 M hydrochloric acid (100 ml) and extracted with ethyl acetate (3×100 ml). The organic extracts were dried over anhydrous magnesium sulfate and evaporated to give an orange solid, which was purified by flash column chromatography eluting with 5% ethyl acetate in isohexane. The subtitle compound was obtained as an orange solid. Yield 410 mg. ¹H NMR CDCl₃: δ 8.48 (1H, br s), 7.87 (1H, dd), 7.22 (1H, m), 6.92 (1H, t), 2.44 (3H, s).

### (iii) 5-Chloro-2-[(3-methyl-4-nitro-1H-indol-7-yl)thio]-1H-benzimidazole

A stirred solution of 5-chloro-1H-benzimidazole-2-thiol (285 mg) in 2-methoxyethanol (6 ml) was treated with aqueous potassium hydroxide solution (0.93 ml, 1.67 M) and stirred at 130° C. for 30 min. 7-Fluoro-3-methyl-4-nitro-1H-indole (300 mg) was added and the solution stirred at 130° C. for 7 h then evaporated in vacuo. The residue was purified by flash column chromatography eluting with 15-30% ethyl acetate in isohexane to give the title compound. Yield 200 mg. M.pt. 252-253° C. ¹H NMR d₆-DMSO: δ 12.75 (1H, br s), 11.88 (1H, br s), 7.77 (1H, d), 7.55-7.35 (4H, br m), 7.17 (1H, dd), 2.31 (3H, s).

### Example 15

### 7-[(5-Chloro-1H-benzimidazol-2-yl)thio]-3-methyl-1H-indol-4-amine

The title compound was prepared from 5-chloro-2-[(3-methyl-4-nitro-1H-indol-7-yl)thio]-1H-benzimidazole (160 mg) according to the method of example 10 step (iv). The product was purified by flash column chromatography eluting with 20-30% ethyl acetate in isohexane. Yield 40 mg. M.pt. 229-230° C. ¹H NMR d₆-DMSO: δ 11.80 (1H, br d), 10.53 (1H, br s), 7.42 (1H, m), 7.25 (1H, d), 7.07 (2H, m), 6.79 (1H, s), 6.25 (1H, d), 5.42 (2H, br s), 2.45 (3H, s).

### Example 16

### 5-Chloro-2-([3-(methylsulfinyl)-4-nitro-1H-indol-7-yl]thio}-1H-benzimidazole

### (i)7-Fluoro-3-(methylsulphinyl)-4-nitro 1H-indole

7-Fluoro-3-(methylthio)-4-nitro-1H-indole (0.25 g) was added to methanol (8 ml) and to this was added a solution of oxone (0.68 g) in water (3 ml). The reaction mixture was stirred at room temperature for 10 min. Methanol was removed from the reaction mixture by evaporation in vacuo and the remaining aqueous mixture was neutralised by addition of saturated sodium bicarbonate solution. The product was extracted into ethyl acetate and insoluble material was removed by filtration. The ethyl acetate was washed with brine, dried over anhydrous magnesium sulfate, filtered and concentrated in vacuo. The material obtained, plus the insoluble material removed earlier, were both found, by LC/MS, to be the desired product, (0.2 g, 83%). ¹H NMR d₆-DMSO: δ 13.30 (1H, br s); 8.21-8.15 (2H, m); 7.32 (1H, t); 2.79 (3H, s).

### (ii) 5-Chloro-2-{[3-(methylsulfinyl)-4-nitro-1H-indol-7-yl]thio}-1H-benzimidazole

5-Chloro-1H-benzimidazole-2-thiol (0.15 g) in N-propanol (5 ml) was treated with aqueous potassium hydroxide solution (0.5 ml, 1.67 M). 7-Fluoro-3-methylsulphinyl)-4-nitro-1H-indole (0.2 g) was then added and the mixture was heated in a sealed tube at 90° C. over night. The reaction mixture was concentrated in vacuo and the residue was purified by flash chromatography, eluting with ethyl acetate containing 0.1% triethylamine. This gave the title compound, 0.2 g (59%). ¹H NMR d₆-DMSO: δ 8.16-8.11 (2H, m); 7.55 (1H, s); 7.49 (2H, dd); 7.20 (1H, dd); 2.82 (3H, s). MS: APCI (+ve) 407(M+1). M.pt. 243-246° C.

### Example 17

### 7-[(5-Chloro-1H-benzimidazol-2-yl)thio]-3-(methylsulfinyl)-1-indol-4-amine

5-Chloro-2-{[3-(methylsulfinyl)-4-nitro-1H-indol-7-yl]thio}-1H-benzimidazole (0.18 g) and iron powder (0.16 g) in ethanol:water:acetic acid:formic acid (70:22:7:1, 10 ml) was heated at 70° C. for 1 h. The mixture was filtered through silica gel and the filtrate concentrated in vacuo. The residue was purified by flash chromatography, eluting with ethyl acetate containing 0.1% triethylamine. This gave the title compound as an off-white solid, (0.06 g. 42%). ¹H NMR d₆-DMSO: δ 12.05 (1H, d); 11.74 (1H, s); 7.64 (1H, d); 7.47 (1H, d); 7.43-7.26 (2H, m); 7.08 (3H, dd); 6.54-6.48 (3H, m); 2.86 (3H, s). MS APCI(+ve) 377(M+1). M.pt. 245-247° C.

### Example 18

### 4-Amino-7-[(5-chloro-1H-benzimidazol-2-yl)thio]-1H-indole-3-carbaldehyde

### (i) 7-Fluoro-3-(methylthio)-4-nitro-indoline

A solution of 7-fluoro-3-(methylthio)-4-nitro-,1,3-dihydro-2H-indol-2-one (0.2 g) in dry tetrahydrofuran (10 ml) was treated with 1 M borane in tetrahydrofuran(1.66 ml). The reaction mixture was stirred at room temperature for twenty h. A further 1.66 ml of 1 M borane solution was added dropwise to the reaction mixture and stirring was continued for one h. The reaction mixture was quenched with a mixture of 2 M hydrochloric acid and methanol and then basified with saturated sodium hydrogen carbonate. The product was extracted into ethyl acetate (×3), dried over anhydrous magnesium sulfate, filtered and evaporated in vacuo. The resulting product was purified by flash chromatography eluting with 20% ethyl acetate in iso-hexane. The sub-title compound was obtained as a yellow oil. Yield 69 mg. (This reaction also gives 7-fluoro-3-(methylthio)-4-nitro-1H-indole as a by-product). ¹H NMR d₆-DMSO: δ 7.42-7.37 (1H, dd); 7.26-7.19 (1H, t); 6.64(1H,s): 4.98-4.95 (1H, d); 4.04-3.97(1H,m); 3.73-3.70 (1H, d); 1.97 (3H,s). MS: APCI (−ve) 227(M−H).

### (ii) 7-Fluoro-4-nitro-1H-indole

7-Fluoro-3-(methylthio)-4-nitro-indoline (650 mg) in methanol (30 ml) was treated with a solution of potassium peroxymonosulfate (3.6 g) in water (30 ml) and the whole was stirred at room temperature for 72 h. The methanol was removed in vacuo and the residue was neutralized with aqueous sodium hydrogen carbonate. The product was extracted into ethyl acetate (3×30 ml) and the combined extracts were washed with saturated sodium metabisulfite solution, followed by brine and then dried over anhydrous magnesium sulfate. After filtration the solvent was removed in vacuo to give the sub-title compound as a light brown solid. Yield 232 mg. MS: APCI (+ve) 179(M+H). ¹H NMR 300 MHz d₆-DMSO: δ 8.14-8.10(1H, dd); 7.84-7.82 (1H, t); 7.22-7.16(1H, t); 7.12-7.10 (1H, m).

### (iii) 7-Fluoro-4-nitro-1H-indole-3-carbaldehyde

Phosphorus oxychloride (84 μl) was cooled to −10° C. in an ice/acetone bath. Dry dimethylformamide (300 μl) was added and the whole was stirred for ten min. 7-Fluoro-4-nitro-1H-indole (150 mg) in dry dimethylformamide (600 μl) was added to the reaction mixture and the whole was allowed to slowly warm to 20° C. After three h stirring at room temperature the reaction was quenched by the addition of ice followed by 2 M sodium hydroxide solution (5 ml). The aqueous solution was extracted with ethyl acetate (10 ml), the pH of the aqeous was adjusted to 6 by addition of dilute hydrochloric acid and then re-extracted with ethyl acetate (20 ml). The combined organic extracts were dried over anhydrous magnesium sulfate and evaporated in vacuo to give the sub-title compound as a dark yellow oil (150 mg). ¹H NMR CDCl₃: δ 10.55(1H, s); 8.27 (1H, s); 8.18-8.15 (1H, m); 7.10-7.04 (1H, m). MS: APCI(−ve) 207 (M−1).

### (iv) 7-[(5-chloro-1H-benzimidazol-2-yl)thio]-4-nitro-1H-indole-3-carbaldehyde

5-Chlorobenzimidazole-2-thiol (200 mg) in N-propanol (10 ml) was treated with potassium hydroxide (0.9 ml, 1.67 M) followed by 7-fluoro-4-nitro-1H-indole-3-carbaldehyde (150 mg). The whole was heated at 90° C. for 15 h, under stirring. The solvent was removed in vacuo, and the residue was taken up into ethyl acetate (20 ml) and washed with sodium bicarbonate solution. The organic phase was dried over anhydrous magnesium sulfate and evaporated in vacuo. The residue was purified using flash chromatography eluting with 50% ethyl acetate in isohexane to give the sub-title compound as an orange solid. Yield 20 mg. MS: APCI(−ve) 371/373 (M−1).

### (v) 4-Amino-7-[(5-chloro-1H-benzimidazol-2-yl)thio]-1H-indole-3-carbaldehyde

7-[(5-Chloro-1H-benzimidazol-2-yl)thio]-4-nitro-1H-indole-3-carbaldehyde (15 mg) and iron powder (180 mg) in ethanol:water:acetic acid:formic acid (70:22:7:1, 10 ml) was stirred under reflux conditions for one h. The mixture was cooled to room temperature and filtered. The filtrate was evaporated in vacuo and the residue was dissolved in ethyl acetate (5 ml) and washed with saturated sodium bicarbonate solution (5 ml) followed by brine (5 ml). The organic phase was dried over anhydrous magnesium sulfate and evaporated in vacuo. The residue was purified using flash chromatography eluting with 50% ethyl acetate in isohexane to give the title compound as a pale yellow solid. Yield 7 mg. ¹H NMR d₆-DMSO: δ 12.22 (1H, br s); 12.08 and 12.04 (1H, 2×br s×2 isomers); 9.70 (1H, s); 8.22 (1H, s); 7.48/7.46 and 7.35/7.33 (1H, 2×d−2 isomers); 7.53 and 7.35 (1H, 2×s−2 isomers); 7.33/7.31(1H, d); 7.15/7.13(1H, d), 6.91 (2H, s); 6.49/6.47 (1H, d). MS: APCI(−ve) 341/343 (M−1).

### Example 19

### 7-[(5-Chloro-1H-benzimidazol-2-yl)thio]-3-(methylsulfonyl)-1H-indole

The title compound was prepared from 5-Chloro-2-methanesulfonyl-1H-benzimidazole (191 mg) and sodium 3-(methylsulfonyl)-1H-indole-7-thiolate according to the method of Example 13 step (v). The product was purified by flash column chromatography eluting with 30% ethyl acetate in isohexane. Yield 28 mg. M.pt 284-286° C. ¹H NMR d₆-DMSO: δ 12.44 (1H, br s), 8.00 (2H, m), 7.59 (1H, d), 7.47-7.32 (3H, m), 7.12 (1H, d), 3.24 (3H, s).

### Example 20

### 4-[(5-chloro-1H-benzimidazol-2-yl)thio]-1,3-dihydro-2H-benzimidazol-2-one

### (i) 4-Hydroxy-1,3-dihydro-2H-benzimidazol-2-one

To a stirred solution of 2,3-diaminophenol (10.34 g) in dimethylformamide (100 ml) was added 1,1′-carbonyldiimidazole (13.48 g) portionwise in order to keep the reaction temperature below 60° C. The solution was then stirred for a further 1 h. The solvent was removed in vacuo and the residue was dissolved in ethyl acetate, and washed with 2 M sodium hydroxide solution (2×). The basic solutions were combined, acidified to pH 2 using 1 M hydrochloric acid, and extracted with ethyl acetate (4×). The ethyl acetate portions were combined, washed with brine, dried over anhydrous magnesium sulfate, filtered and concentrated in vacuo to give a brown solid. The solid was washed with ether to afford the sub-title compound. Yield 6 g. ¹H NMR d₆-DMSO: δ10.42 (1H, s); 10.31 (1H, s); 9.46 (1H.s); 6.72 (1H,t); 6.44-6.40 (2H,m).

### (ii) O-(2-oxo-2,3-dihydro-1H-benzimidazol-4-yl) dimethylthiocarbamate

Dimethylthiocarbamoyl chloride (2.46 g) in dimethylformamide (20 ml) was added dropwise to a solution of the 4-hydroxy-1,3-dihydro-2H-benzimidazol-2-one (3 g) and cesium carbonate (6.5 g) in dimethylformamide(100 ml. The reaction was stirred for 2 h before the solvent was removed in vacuo and the residue partitioned between water and ethyl acetate. The organic phase was washed with 2 M sodium carbonate solution and saturated brine, dried over anhydrous magnesium sulfate and concentrated in vacuo to give a solid. This was triturated with acetonitrile to afford the sub-title compound. Yield 1 g. ¹H NMR d₆-DMSO: δ 10.90 (1H, s); 10.72 (1H,s); 6.90 (1H, t); 6.80 (1H, d); 6.63(1H, d); 3.36 (3H, s); 3.30 (3H, s).

### (iii) S-(2-oxo-2,3-dihydro-1H-benzimidazolyl) dimethylthiocarbamate

A stirred suspension of O-(2-oxo-2,3-dihydro-1H-benzimidazolyl) dimethylthiocarbamate (0.9 g) in diphenyl ether (10 ml) was heated at reflux temperature for 2 h. The cooled suspension was added to isohexane (200 ml) and the precipitate was isolated by filtration. The solid was washed with ethylacetate and acetonitrile and the combined washings were concentrated in vacuo. The resulting solid was purified by flash column chromatography eluting with 5% methanol in dichloromethane to give the sub-title compound. Yield 0.2 g. 1H NMR d6-DMSO: (Rotamers) δ 10.90-10.69 (2H, m); 7.01-6.65 (3H, m); 3.08-2.87 (6H, m).

### (iv) 4-mercapto-1,3-dihydro-2H-benzimidazol-2-one

12 M Sodium hydroxide (0.27 ml) was added to a suspension of S-(2-oxo-2,3-dihydro-1H-benzimidazol-4-yl) dimethylthiocarbamate (0.155 g) in water (5 ml) and heated to reflux temperature for 2 h. The solution was cooled to room temperature and acetic acid added to adjust the pH to 5. The precipitated solid was isolated by filtration to give the sub-titled compound. Yield 0.076 g. MS: APCI(+ve) 167(M+1).

### (v) 4-[(5-chloro-1H-benzimidazol-2-yl)thio]-1,3-dihydro-2H-benzimidazol-2-one

To a stirred solution of 5-chloro-2-(methylsulfonyl)-1H-benzimidazole (0.106 g) in isopropanol (10 ml) was added 4-mercapto-1,3-dihydro-2H-benzimidazol-2-one (0.076 g). The mixture was heated at reflux temperature for 18 h. The solvent was removed in vacuo and the residue purified by reverse phase HPLC eluting with 75-05% 0.1% NH₃(aq)/acetonitrile to give titled compound. Yield 0.030 g. ¹H NMR d₆-DMSO: δ 12.39 (1H, br s); 11.04 (1H,br s); 10.90 (1H, s); 7.45 (1H, br s); 7.40(1H, br s); 7.13 (2H, d); 7.08-6.99 (2H, m). MS: APCI(+ve) 317 (M+1).

### Example 21

### 7-[(5-Chloro-1H-benzimidazol-2-yl)thio]-2-oxo-2,3-dihydro-1H-benzimidazole-5-carboxylic Acid

### (i)7-(Chlorosulfonyl)-2-oxo-2,3-dihydro-1H-benzimidazole-5-carboxylic Acid

Chlorosulfonic acid (4 ml) was added carefully to 2-oxo-2,3-dihydro-1H-benzimidazole-5-carboxylic acid methyl ester (1.0 g) (ref Justus Liebigs Ann. Chem.; 1896 (291); 328) and the mixture was heated at 100° C. for 8 h. The reaction mixture was cooled to room temperature and carefully added dropwise into cold methanol (30 ml, −40° C.). After addition was complete, water (10 ml) was added and product was extracted with ethyl acetate (×3). The ethyl acetate was washed with brine, dried over anhydrous magnesium sulphate, filtered and concentrated in vacuo to give the sub-title product as a pale brown solid, 1.2 g (83%). ¹H NMR d₆-DMSO: δ 13.43 (1H, br s); 10.92 (1H, s); 9.72 (1H, s); 7.82 (1H, s); 7.41 (1H, s).

### (ii) 7-Mercapto-2-oxo-2,3-dihydro-1H-benzimidazole-5-carboxylic Acid

7-(Chlorosulfonyl)-2-oxo-2,3-dihydro-1H-benzimidazole-5-carboxylic acid (0.5 g) was added to tetrahydrofuran (10 ml) and the resulting mixture was treated, portionwise, with triphenylphosphine (1.66 g). Water (2 ml) was then added and the reaction mixture was heated at 50° C. for 1 h. The reaction was cooled to room temperature and water was added followed by 2 M sodium hydroxide solution (20 ml). The mixture was washed with ethyl acetate and then acidified by addition of 2 M hydrochloric acid (20 ml). The product was extracted with ethyl acetate, dried over anhydrous sodium sulfate, filtered and concentrated in vacuo, to give the desired product, (0.3 g, 79%). ¹H NMR d₆-DMSO: δ 12.45 (1H, br s); 11.11 (1H, s); 11.04 (1H, s); 7.55 (1H, s); 7.28 (1H, s); 5.38 (1H, br s).

### (iii) 7-[(5-Chloro-1H-benzimidazol-2-yl)thio]-2-oxo-2,3-dihydro-1H-benzimidazole-5-carboxylic Acid

The title compound was prepared from 7-mercapto-2-oxo-2,3-dihydro-1H-benzimidazole-5-carboxylic acid (91 mg) and 5chloro-2-methanesulfonyl-1H-benzimidazole (100 mg) by the method described in example 20 (v). Yield 70 mg. ¹H NMR d₆-DMSO: δ 11.51 (1H, br s); 11.20 (1H, br s); 7.79 (1H, br s); 7.57 (2H, dd); 7.46 (1H, dd); 7.22 (1H, dd); 2.35 (2H, s). M.pt. 326-330° C.

### Example 22

### 7-{[5Chloro-4-(methoxycarbonyl)-1H-benzimidazol-2-yl]thio-}-2-oxo-2,3-dihydro-1H-benzimidazole-5-carboxylic Acid

The title compound was prepared from 7-mercapto-2-oxo-2,3-dihydro-1H-benzimidazole-5-carboxylic acid (74 mg) and methyl 5-chloro-2-(methanesulfonyl)-1H-benzimidazole-4-carboxylate (100 mg) by the method described in example 20 (v). Yield 75 mg. ¹H NMR d₆-DMSO: δ 12.86 (1H, br s); 11.43 (1H, br s); 11.14 (1H, br s); 7.78 (1H, br s); 7.57 (2H, br s); 7.24 (1H, d); 3.94 (3H, s).

### Example 23

### 7-({5-Chloro-4-[(2-hydroxyethoxy)carbonyl]-1H-benzimidazol-2-yl}thio)-2-oxo-2,3-dihydro-1H-benzimidazole-5-carboxylic Acid

### (i) 5-Chloro-2-(methylthio)-1H-benzimidazole-4-carboxylic Acid

5-Chloro-2-(methylthio)-1H-benzimidazole-4-carbonitrile (2.4 g) in 10 M sodium hydroxide (40 ml) was refluxed for 16 h. The reaction was cooled and quenched with 2 M hydrochloric acid until it reached pH 7 and a precipitate formed. The carboxylic acid was isolated by filtration and air dried to give a brown solid (2 g). MS: APCI(−ve) 242 (M−1).

### (ii) 2-Hydroxyethyl 5-chloro-2-(methylthio)-1H-benzimidazole-4-carboxylate

5-Chloro-2-(methylthio)-1H-benzimidazole-4-carboxylic acid (2 g) was suspended in thionyl chloride (50 ml) and refluxed for 2 h. The excess thionyl chloride was removed in vacuo and the solid residue treated with ethylene glycol (20 ml). The mixture was heated at 80° C. for two h then cooled and partitioned between ethyl acetate and water. The ester was isolated by drying the organic layer and removing the solvent in vacuo. MS: APCI(−ve) 286 (M−1).

### (iii) 2-Hydroxyethyl 5-chloro-2-(methylsulfonyl)-1H-benzimidazole-4-carboxylate

2-Hydroxyethyl 5-chloro-2-(methylthio)-1H-benzimidazole-4-carboxylate (1 g) in methanol (10 ml) was treated with oxone (4.68 g) in water (10 ml) and stirred over night.

The methanol was removed in vacuo and the reaction mixture treated with aqueous sodium bicarbonate. The required product was extracted with ethyl acetate, dried and the solvent removed in vacuo to give 2-hydroxyethyl 5-chloro-2-(methylsulfonyl)-1H-benzimidazole-4-carboxylate (1 g). MS: APCI(−ve) 317 (M−1).

(iv) 7-({5-Chloro-4-[(2-hydroxyethoxy)carbonyl]-1H-benzimidazol-2-yl}thio)-2-oxo-2,3-dihydro-1H-benzimidazole-5-carboxylic acid. The title compound was prepared from 2-hydroxyethyl 5-chloro-2-(methylsulfonyl)-1H-benzimidazole-4-carboxylate (0.16 g) and 7-mercapto-2-oxo-2,3-dihydro-1H-benzimidazole-5-carboxylic acid (0.16 g) by refluxing them together in isopropanol for 4 h. Upon cooling the product (0.1 g) was isolated by filtration. ¹H NMR d₆-DMSO: δ 11.45 (s, 1H), 11.15 (s, 1H), 7.8 (s, 1H), 5.56 (s, 1H), 7.55 (s, 1H), 4.4 (t, 2H), 3.71 (t, 3H).

### Example 24

### Methyl 7-[(5-chloro-1H-benzimidazol-2-yl)thio]-2-oxo-2,3-dihydro-1H-benzimidazole-5-carboxylate

Prepared as described in Method 1.

### (i) 7-(Chlorosulfonyl)-2-oxo-2,3-dihydro-1H-benzimidazole-5-carboxylic Acid

Chlorosulfonic acid (4 ml) was added carefully to 2-oxo-2,3-dihydro-1H-benzimidazole-5-carboxylic acid methyl ester (1.0 g) (ref. Justus Liebigs Ann. Chem.; 1896 (291); 328) and the mixture was heated at 100° C. for 8 h. The reaction mixture was cooled to room temperature and carefully added dropwise into cold methanol (30 ml, −40° C.). After addition was complete, water (10 ml) was added and the product was extracted with ethyl acetate (×3). The ethyl acetate was washed with brine, dried over anhydrous magnesium sulphate, filtered and concentrated in vacuo to give the sub-title product as a pale brown solid, 1.2 g (83%). ¹H NMR d₆-DMSO: δ 13.43 (1H, br s); 10.92 (1H, s); 9.72 (1H, s); 7.82 (1H, s); 7.41 (1H, s).

### (ii) 7-Mercapto-2-oxo-2,3-dihydro-1H-benzimidazole-5-carboxylic Acid

7-(Chlorosulfonyl)-2-oxo-2,3-dihydro-1H-benzimidazole-5-carboxylic acid (0.5 g) was added to tetrahydrofuran (10 ml) and the resulting mixture was treated, portionwise, with triphenylphosphine (1.66 g). Water (2 ml) was then added and the reaction mixture was heated at 50° C. for 1 h. The reaction mixture was cooled to room temperature and water was added followed by 2 M sodium hydroxide solution (20 ml). The mixture was washed with ethyl acetate and then acidified by addition of 2 M hydrochloric acid (20 ml). The product was extracted with ethyl acetate, dried over anhydrous sodium sulfate, filtered and concentrated in vacuo, to give the desired product, (0.3 g, 79%). ¹H NMR d₆-DMSO: δ 12.45 (1H, br s); 11.11 (1H, s); 11.04 (1H, s); 7.55 (1H, s); 7.28 (1H, s); 5.38 (1H, br s).

### (iii) 7-[(5-Chloro-1H-benzimidazol-2-yl)thio]-2-oxo-2,3 dihydro-1H-benzimidazole-5-carboxylic Acid

To a stirred solution of 5-chloro-2-(methylsulfonyl)-1H-benzimidazole (100 mg) (described in U.S. Pat. No. 3,480,643), in isopropanol (10 ml) was added 7-mercapto-2-oxo-2,3-dihydro-1H-benzimidazole-5-carboxylic acid (91 mg). The mixture was heated at reflux temperature for 18 h. The solvent was removed in vacuo and the residue purified by reversed phase HPLC eluting with a gradient of 0.1% NH₃(aq)/acetonitrile to give titled compound. Yield 70 mg. ¹H NMR d₆-DMSO: δ 11.51 (1H, br s); 11.20 (1H, br s); 7.79 (1H, br s); 7.57 (2H, dd); 7.46 (1H, dd); 7.22 (1H, dd); 2.35 (2H, s).

### (iv) Methyl 7-[(5-chloro-1H-benzimidazol-2-yl)thio]-2-oxo-2,3-dihydro-1H-benzimidazole-5-carboxylate

Dowex H⁺ was added to a solution of 7-[(5-chloro-1H-benzimidazol-2-yl)thio]-2-oxo-2,3-dihydro-1H-benzimidazole-5-carboxylic acid (100 mg, 0.28 mmol) in methanol (50 ml). The reaction mixture was refluxed over night, thereafter the Dowex H⁺ was filtered off, washed with DMF and CH₂Cl₂. The filtrate was concentrated and purified by flash column chromatography (CH₂Cl₂/MeOH 5:1) to give 12 mg (12%) of the title compound. ¹H NMR (DMSO-d₆): δ 3.81 (3H, s), 7.12 (1H, dd), 7.40 (1H, m), 7.47 (1H, m), 7.55 (1H, d), 7,77 (1H, d), 10.75 (1H, s), 11.18 (1H, s), 12.60 (1H, s), ¹³C NMR (DMSO-d₆): δ 52.2, 106.7, 108.4, 109.5, 110.1, 120.1, 121.8, 122.6, 124.5, 129.0, 130.4, 130.8, 137.2, 155.2, 165.7. MS (EC1) m/z 375 and 377 (M+1).

### Example 25

### Ethyl 7-[(5-chloro-1H-benzimidazol-2-yl)thio]-2-oxo-2,3-dihydro-1H-benzimidazole 5-carboxylate

A drop of H₂SO₄ was added to a solution of 7-[(5-chloro-1H-benzimidazol-2-yl)thio]-2-oxo-2,3-dihydro-1H-benzimidazole-5-carboxylic acid (95 mg, 0.26 mmol) in ethanol (20 ml). The reaction was refluxed over night, neutralized, concentrated and purified by precipitation from a MeOH/CH₂Cl₂ (1:3) solution to give 56 mg (55%) of the title compound. ¹H NMR (DMSO-d₆): δ 1.29, (3H, tr), 4.27 (2H, q), 7.12 (1H, d), 7.42 (2H, m), 7.55 (1H, d), 7.76 (1H, d), 11.18 (1H, s), 11.53 (1H, s), 12.53 (1H, s). ¹³C NMR (DMSO-d₆): δ 14.2, 60.8, 106.6, 110.2, 121.8, 122.9, 129.0, 130.4, 137.2, 155.2, 165.1. MS (ECI) m/z 389 and 391 (M+1).

### Example 26

### 2-Morpholin-4-ylethyl 7-[(5-chloro-1H-benzimidazol-2-yl)thio)-2-oxo-2,3-dihydro-1H-benzimidazole-5-carboxylate

7-[(5-chloro-1H-benzimidazol-2-yl)thio]-2-oxo-2,3-dihydro-1H-benzimidazole-5-carboxylic acid (100 mg, 0.27 mmol) was dissolved in 2 ml dry DMF. N-hydroxyethylmorpholine (327 μl, 2.7 mmol), HATU (308 mg, 0.81 mmol), and N,N-diisopropyldiethyl amine (285 μl, 1.6 mmol) were added and the resulting mixture was stirred for 6 days at 60° C. The solvent was removed and the resulting yellow solid was purified by preparative reversed phase HPLC to yield 33 mg (26%) of the title compound. ¹H NMR (DMSO-d₆): δ 2.34, (4H, m), 2.69 (2H, m), 3.61 (4H, m), 4.35 (2H, m), 7.13 (1H, d), 7.33 (1H, d), 7.48 (1H, d), 7.57 (1H, s), 7.98 (1H, s). ¹³C NMR (DMSO-d6): δ 53.7, 56.8, 62.3, 66.5, 107.3, 110.5, 122.2, 123.1, 126.1 129.1, 130.8, 134.6, 137.4, 151.2, 155.6, 159.3, 165.5. MS (ECI) m/z 474 (M+1).

### Example 27

### 7-[(5-Chloro-6-fluoro-1H-benzimidazol-2-yl)thio]-2-oxo-2,3-dihydro-1H-benzimidazole-5-carboxylic Acid

Prepared as described in Method 1.

5-chloro-6-fluoro-2-(methylsulfonyl)1H-benzimidazole (0.53 g, 2.14 mmol) and 7-mercapto-2-oxo-2,3-dihydro-1H-benzimidazole-5-carboxylic acid (0.45 g, 2.14 mmol) dissolved in a THF/2-propanol solution (30 ml, 1:1) were refluxed for 2 h and then concentrated in vacuo. The crude mixture was partitioned between a 1 M NaOH (aq) solution and CH₂Cl₂. The aqueous phase was pH adjusted to approximately pH 3. The precipitate was filtered off, washed with water and dried. The solid was then refluxed in a CH₂Cl₂/MeOH solution (3:1), filtered off and washed with CH₂Cl₂ to give after drying 0.79 g (97%) of the title compound. ¹H NMR (DMSO-d₆): δ 7.53 (2H, m), 7.56 (1H, s), 7.74 (1H, s), 11.23 (1H, s), 11.46 (1H, s), 12.78 (1H, s). MS (ECI) m/z 379 and 381 (M+1).

### Example 28

### 7-[(5-Methoxy-1H-benzimidazol-2-yl)thio]-2-oxo-2,3-dihydro-1H-benzimidazole-5-carboxylic Acid

Prepared as described in Method 1. Starting from 5-methoxy-2-(methylsulfonyl)-1H-benzimidazole (0.48 g, 2.14 mmol) and 7-mercapto-2-oxo-2,3-dihydro-1H-benzimidazole-5-carboxylic acid (0.45 g, 2.14 mmol) to give 0.75 g (98%) of the title compound. ¹H NMR (DMSO-d6): δ 3.75 (3H, s), 6.81 (1H, dd), 6.96 (1H, s), 7.36 (1H, d), 7.54 (1H, s), 7.72 (1H, s), 11.19 (1H, s), 11.50 (1H, s). MS (ECI) m/z 357 (M+1).

### Example 29

### 7-[(5-Bromo-1H-benzimidazol-2-yl)thio]-2-oxo-2,3-dihydro-1H-benzimidazole-5-carboxylic Acid

Prepared as described in Method 1. Starting from 5-bromo-2-(methylsulfonyl)-1H-benzimidazole (344 mg, 1.25 mmol) and 7-mercapto-2-oxo-2,3-dihydro-1H-benzimidazole-5-carboxylic acid (263 mg, 125 mmol) to give 344 mg (68%) of the title compound. ¹H NMR (DMSO-d6): δ 7.64 (2H, dd), 7.89 (1H, s), 7.96 (1H, s), 8.04 (1H, s), 11.49 (1H, s), 11.80 (1H, s). MS (ECI) m/z 406 (M+1).

### Example 30

### 7-[(4-Bromo-6-fluoro-1H-benzimidazol-2-yl)thio]-2-oxo-2,3-dihydro-1H-benzimidazole-5-carboxylic Acid

Prepared as described in Method 1. Starting from 4-bromo-6-fluro-2-(methylsulfonyl)-1H-benzimidazole (337 mg, 1.15 mmol) and 7-mercapto-2-oxo-2,3-dihydro-1H-benzimidazole-5-carboxylic acid (263 g, 1.25 mmol) to give 369 mg (76%) of the title compound. ¹H NMR (DMSO-d6): δ 7.34 (1H, s), 7.42 (1H, s), 7.54 (1H, s), 7.62 (1H, s), 11.09 (1H, s), 11.43 (1H, s). MS (ECI) m/z 424 (M+1).

### Example 31

### 7-[(5-Chloro-1H-benzimidazol-2-yl)thio]-N-[2-(4-morpholin-4-ylethyl]-2-oxo-2,3-dihydro-1H-benzimidazole-5-carboxamide

Prepared as described in Method 2.

Hünig's base (47 μl, 0.27 mmol) was added to a stirred solution of 7-[(5-chloro-1H-benzimidazol-2-yl)thio]-2-oxo-2,3-dihydro-1H-benzimidazole-5-carboxylic acid (90 mg, 0.25 mmol) and 4-(2-aminoethyl)morpholine (36 μl, 0.27 mmol) in DMF (4.0 ml) at ambient temperature. Then TBTU (88 mg, 0.27 mmol), HOBTxH₂O (34 mg, 0.25 mmol) and Hünig's base (47 μl, 0.27 mmol) were added. After stirring for 2.5 h the reaction mixture was concentrated in vacuo and purified by flash column chromatography (CH₂Cl₂/MeOH 5:1→2:1) to give 95 mg (81%) of the title compound. ¹H NMR (DMSO-d6): δ 2.38 (4H, m), 2.42 (2H, tr), 3.35 (2H, m), 3.53 (4H, tr), 7.13 (1H, d), 7.35 (1H, m), 7.47 (1H, m), 7.55 (1H, s), 7.73 (1H, s), 8.41 (1H, tr), 11.15 (1H, s), 11.26 (1H, s), 12.45 (1H, s). MS (ECI) m/z 473 and 475 (M+1).

### Example 32

### 7-[(5-Chloro-1H-benzimidazol-2-yl)thio]-N-[3-dimethylamino)ethyl]-2-oxo-2,3-dihydro-1H-benzimidazole-5-carboxamide

Method 2:

Hünig's base (30 pd, 0.17 mmol) was added to a slurry of 7-[(5-chloro-1H-benzimidazol-2-yl)thio]-2-oxo-2,3 dihydro-1H-benzimidazole-5-carboxylic acid (57 mg, 0.14 mmol) and N,N-dimethylethylenediamine (17 μl, 0.17 mmol) in a DMF/acetonitrile (4.0 ml, 1:3) solution at ambient temperature. After 10 min TBTU (56 mg, 0.17 mmol), HOBTxH₂O (21 mg, 0.17 mmol) and Hünig's base (30 μl, 0.17 mmol) were added. An additional amount of N. N-dimethylethylenediamine, TBTU, HOBT xH₂O and Hünig's base were added after 4 h and the reaction mixture was stirred over night, then concentrated in vacuo and purified by flash column chromatography (CH₂Cl₂/MeOH 2:1→1:3) to give 58 mg (86%) of the title compound. ¹H NMR (DMSO-d6): δ 2.14 (6H, s), 2.35 (2H, tr), 3.30 (2H, q), 7.06 (1H, dd), 7.36 (1H, d), 7.42 (1H, d), 7.52 (1H, s), 7.71 (1H, s), 8.37 (1H, tr). MS (ECI) m/z 431 and 433 (M+1).

### Example 33

### 4-[(5-Chloro-1H-benzimidazol-2-yl)thio]-6-[(4-methylpiperazin-1-yl)carbonyl]-1,3-dihydro-2H-benzimidazol-2-one

Method 2:

Hünig's base (108 μl, 0.62 mmol) was added to a solution of 7-[(5-chloro-1H-benzimidazol-2-yl)thio]-2-oxo-2,3-dihydro-1H-benzimidazole-5-carboxylic acid (150 mg, 0.42 mmol) and N-methylpiperazine (51 μl, 0.46 mmol) in DMF (4.0 ml) at ambient temperature. After 5 min TBTU (147 mg, 0.46 mmol), HOBTxH₂O (56 mg, 0.42 mmol) and Hünig's base (85 μl, 0.46 mmol) were added. After stirring for 3 h the reaction mixture was concentrated in vacuo and purified twice by flash column chromatography (CH₂Cl₂/MeOH 5:1→2:1) to give 51 mg (28%) of the title compound. ¹H NMR (DMSO-d6): δ 2.14 (3H, s), 2.22 (4H, m), 3.45 (4H, m), 7.03 (1H, d), 7.08 (1H, d), 7.14 (1H, dd), 7.41 (1H, m), 7.48 (1H, m), 11.07 (1H, s), 11.30 (1H, s), 12.66 (1H, s). MS (ECI) m/z 443 and 445 (M+1).

### Example 34

### 7-[(5-Chloro-1H-benzimidazol-2-yl)thio]-N-methyl-2-oxo-2,3-dihydro-1H-benzimidazole-5-carboxamide

Methylamine 8.0 M solution in ethanol (76 μl, 0.61 mmol) was added to a solution of 7-[(5-chloro-1H-benzimidazol-2-yl)thio]-2-oxo-2,3-dihydro-1H-benzimidazole-5-carboxylic acid (200 mg, 0.55 mmol) and Hünig's base (144 μl, 0.83 mmol) in DMF (4.0 ml) at ambient temperature. After 5 min TBTU (147 mg, 0.61 mmol), HOBTxH₂O (56 mg, 0.55 mmol) and Hünig's base (106 μl, 0.61 mmol) were added. The reaction mixture was stirred over night, concentrated in vacuo and then precipitated from a CH₂Cl₂/MeOH solution to give 102 mg (49%) of the title compound. ¹H NMR (DMSO-d6): δ 2.73 (3H, d), 7.12 (1H, d), 7.39 (2H, m), 7.55 (1H, s), 7.72 (1H, s), 8.42 (1H, d), 11.16 (1H, s), 11.34 (1H, s), 12.44 (1H, s). MS (ECI) m/z 374 and 376 (M+1).

### Example 35

### 7-[(5-Chloro-1H-benzimidazol-2-yl)thio]-N-(2-hydroxyethyl)-2-oxo-2,3-dihydro-1H-benzimidazole-5-carboxamide

Hünig's base (106 μl, 0.61 mmol) was added to a solution of 7-[(5-chloro-1H-benzimidazol-2-yl)thio]-2-oxo-2,3-dihydro-1H-benzimidazole-5-carboxylic acid (200 mg, 0.55 mmol) and ethanolamine (37 μl, 0.61 mmol) in DMF (5.0 ml) at ambient temperature. After 5 min TBTU (196 mg, 0.61 mmol), HOBTxH₂O (75 mg, 0.55 mmol) and Hünig's base (106 μl, 0.61 mmol) were added. The reaction mixture was concentrated after 2 h and purified by precipitation from a CH₂Cl₂/MeOH solution to give 115 mg (51%) of the title compound. ¹H NMR (DMSO-d6): δ 3.29 (2H, q), 3.49 (2H, tr), 7.10 (1H, d), 7.38 (1H, m), 7.45 (1H, m), 7.59 (1H, s), 7.77 (1H, s), 11.19 (1H, s), 11.32 (1H, s), 12.43 (1H, r s). MS (ECI) m/z 404 and 406 (M+1).

### Example 36

### 4-[(5-Chloro-1H-benzimidazol-2-yl)thio]-6-(piperazin-1-ylcarbonyl)-1,3-dihydro-2H-benzimidazol-2-one Dihydrochloride

Hünig's base (158 μl, 0.92 mmol) was added to a solution of 7-[(5-chloro-1H-benzimidazol-2-yl)thio]-2-oxo-2,3-dihydro-1H-benzimidazole-5-carboxylic acid (300 mg, 0.83 mmol) and tert-butyl 1-piperazinecarboxylate (170 mg, 0.92 mmol) in DMF (6.0 ml) at ambient temperature. After 5 min TBTU (294 mg, 0.92 mmol), HOBTxH₂O (112 mg, 0.83 mmol) and Hünig's base (158 μl, 0.92 mmol) were added. The reaction mixture was concentrated after 2 h and purified twice by flash chromatography (CH₂Cl₂/MeOH 10:1) to give 233 mg (54%) of the boc-protected product. The base was then dissolved in methanol (8 ml) and treated over night with 4 M HCl in diethyl ether (1 ml), concentrated, washed with methanol/diethyl ether (1:1) to give 185 mg (44%) of the title compound. ¹H NMR (DMSO-d₆): δ 3.09 (4H, m), 3.73 (4H, m), 7.16 (1H, d), 7.23 (1H, dd), 7.31 (1H, d), 7.48. (1H, d), 7.55 (1H, d), 9.39 (2H, s), 11.22 (1H, s), 11.36 (1H, s).

### Example 37

### 7-[(5-Chloro-1H-benzimidazol-2-yl) thio]-2-oxo-2,3-dihydro-1H-benzimidazole-5-carboxamide

Hünig's base (144 μl, 0.83 mmol) was added to a solution of 7-[(5-chloro-1H-benzimidazol-2-yl)thio]-2-oxo-2,3-dihydro-1H-benzimidazole-5-carboxylic acid (200 mg, 0.55 mmol) and ammonium chloride (89 mg, 1.7 mmol) in DMF (4.0 ml) at ambient temperature. After 5 min TBTU (196 mg, 0.61 mmol), HOBTxH₂O (75 mg, 0.55 mmol) and Hünig's base (192 μl, 1.1 mmol) were added. The reaction mixture was concentrated after 2 h and purified by a short silica gel column (CH₂Cl₂/MeOH 5:1), followed by precipitation of the concentrated product from a warm MeOH solution to give 70 mg (35%) of the title compound. ¹NMR (DMSO-d6): δ 7.12 (1H, dd), 7.24 (1H, s), 7.42 (2H, m), 7.59 (1H, s), 7.78 (1H, s), 7.97 (1H, s), 11.14 (1H, s), 11.29 (1H, s), 12.52 (1H, s). ¹³C NMR: δ 109.4, 121.6, 127.2, 127.6, 130.2, 135.7, 155.3, 167.0. MS (ECI) m/z 360 and 362 (M+1).

### Example 38

### 7-[(5-Chloro-1H-benzimidazol-2-yl)thio]-N-[2-(2-hydroxyethoxy)ethyl]-2-oxo-2,3-dihydro-1H-benzimidazole-5-carboxamide

TBTU (104 mg, 0.32 mmol), HOBTxH₂O (44 mg, 0.32 mmol) and Hünig's base (57 μl, 0.32 mmol) were added to a solution of 7-[(5-chloro-1H-benzimidazol-2-yl)thio]-2-oxo-2,3-dihydro-1H-benzimidazole-5-carboxylic acid (100 mg, 0.28 mmol) in DMF (3 ml) at ambient temperature. After 5 min 2(2-aminoetoxy)ethanol (33 μl, 0.32 mmol) was added and the reaction was concentrated after 15 min. The crude mixture was purified by precipitation from a methanol-ethyl acetate (1:4) solution to give 14 mg (11%) of the title compound. ¹H NMR (DMSO-d6): δ 3.40 (4H, m), 3.48 (4H, m), 7.11 (1H, d), 7.42 (2H, m), 7.57 (1H,s), 7.75 (1H, s), 8.52 (1H, tr), 11.15 (1H, s), 11.33 (1H, s), 12.43 (1H, s). MS (ECI) m/z 448 and 450 (M+1).

### Example 39

### 4-[(S-Chloro-1H-benzimidazol-2-yl)thio]-6-{[4-(2-hydroxyethyl)piperazin-1-yl]carbonyl]}-1,3-dihydro-2H-benzimidazol-2-one

TBTU (104 mg, 0.32 mmol), HOBTxH₂O (44 mg, 0.32 mmol) and Hünig's base (57 μl, 0.32 mmol) were added to a solution of 7-[(5-chloro-1H-benzimidazol-2-yl)thio)-2-oxo-2,3-dihydro-1H-benzimidazole-5-carboxylic acid (100 mg, 0.28 mmol) in DMF (3 ml) at ambient temperature. After 5 min 1-(2-hydroxyethyl)piperazine (33 μl, 0.32 mmol) was added and the reaction mixture was concentrated after 20 min. The crude mixture was purified by precipitation from a methanol-ethyl acetate (1:3) solution to give 46 mg (35%) of the title compound. ¹H NMR (DMSO-d6): δ 3.03 (4H, m), 3.34 (2H, m), 3.65 (2H, m), 7.10 (1H, s), 7.14 (1H, m), 7.37 (2H, m), 7.48 (1H, m), 11.11 (1H, s), 11.33 (1H, s), 12.57 (1H, s). ¹³C NMR (DMSO-d6): δ 41.7, 51.4, 54.9, 58.3, 109.1, 111.8, 121.9, 125.8, 130.7, 134.3, 155.8, 168.5. MS (ECI) m/z 375 and 377 (M+1).

### Example 40

### 7-[(5-Chloro-1H-benzimidazol-2-yl)thio]-2-oxo-N-4-piperidin-4-yl-2,3-dihydro-1H-benzimidazole-5-carboxamide Dihydrochloride

TBTU (104 mg, 0.33 nmol), HOBTxH₂O (44 mg, 0.32 mmol) and Hünig's base (57 μl, 0.32 mmol) were added to a solution of 7-[(5-chloro-1H-benzimidazol-2-yl)thio]-2-oxo-2,3-dihydro-1H-benzimidazole-5-carboxylic acid (100 mg, 0.28 mmol) in DMF (3 ml) at ambient temperature. After 5 min tert-butyl 4-aminopiperidine-1-carboxylate (67 mg, 0.32-mmol) was added. The reaction mixture was stirred over night and concentrated. The crude mixture was dissolved in methanol/diethyl ether (1:1, 2 ml) and treated over night with 4 M HCl in diethyl ether (1 ml), concentrated and purified by precipitation from a methanol/diethyl ether (1:4) solution to give 37 mg (26%) of the title compound. ¹H NMR (DMSO-d6): δ 1.75 (2H, m), 1.90 (2H, m), 2.95 (2H, m), 3.26 (2H, m), 4.03 (1H, m), 7.20 (1H, dd), 7.45 (1H, d), 7.51 (1H, d), 7.62 (1H, s), 7.84 (1H, s), 8.52 (1H, d), 8.96 (2H, s), 11.21 (1H, s), 11.37 (1H, s). ¹³C NMR (DMSO-d6): δ 28.1, 42.2, 44.5, 104.6, 109.7, 122.4, 126.6, 127.6, 130.4, 135.9, 150.4, 155.3, 164.8. MS (ECI) m/z 443 and 445 (M+1).

### Example 41

### N-(3-Aminopropyl)-7-[(5-chloro-1H-benzimidazol-2-yl)thio]-2-oxo-2,3-dihydro-1H-benzimidazole-5-carboxamide Dihydrochloride

TBTU (104 mg, 0.32 mmol), HOBTxH₂O (44 mg, 0.32 mmol) and Hünig's base (57 μl, 0.32 mmol) were added to a solution of 7-[(5-chloro-1H-benzimidazol-2-yl)thio]-2-oxo-2,3-dihydro-1H-benzimidazole-5-carboxylic acid (100 mg, 0.28 mmol) in DMF (3 ml) at ambient temperature. After 5 min tert-butyl N-(3-aminopropyl)carbamate (58 mg, 0.32 mmol) was added and the reaction mixture was stirred over night, then concentrated. The crude mixture was purified by precipitation from a methanol-dichloromethane (1:1) solution. The dried precipitate was then treated over night with 4 M HCl in diethyl ether (1 ml), then concentrated, washed with diethyl ether to give 63 mg (46%) of the title compound. ¹H NMR (DMSO-d6): δ 1.79 (2H, m), 2.79 (2H, m), 3.30 (2H, m), 7.21 (1H, dd), 7.46 (1H, d), 7.53 (1H, d), 7.62 (1H, d), 7.82 (1H, d), 7.96 (3H, s), 8.74 (1H, tr), 11.27 (1H, s), 11.40 (1H, s). MS (ECI) m/z 417 and 419 (M+1).

### Example 42

### 7-[(5-Chloro-1H-benzimidazol-2-yl)]thio-2-oxo-N—P-(2-oxopyrrolidin-1-yl)propyl]-2,3-dihydro-1H-benzimidazole-5-carboxamide

TBTU (104 mg, 0.32 mmol), HOBTxH₂O (44 mg, 0.32 mmol) and Hünig's base (57 μl, 0.32 mmol) were added to a solution of 7-[(5-chloro-1H-benzimidazol-2-yl)thio]-2-oxo-2,3-dihydro-1H-benzimidazole-5-carboxylic acid (100 mg, 0.28 mmol) in DMF (3 ml) at ambient temperature. After 5 min 1-(3-aminopropyl)-2-pyrrolidinone (47 μl, 0.32 mmol) was added and the reaction mixture was stirred over night. The reaction mixture was then concentrated and purified by precipitation from a methanol-dichloromethane (1:2) solution to give 73 mg (54%) of the title compound. ¹H NMR (DMSO-d6): δ 1.68 (2H, m), 1.89 (2H, m), 2.19 (2H, tr), 3.19 (4H, m), 3.32, (2H, m), 7.1 (1H, m), 7.35 (1H, m), 7.46 (1H, m), 7.56 (1H, s), 7.73 (1H, s), 11.11 (1H, s), 11.33 (1H, s), 12.57 (1H, s). ¹³C NMR (DMSO-d₆): δ 17.5, 26.8, 30.4, 36.9, 46.3 109.4, 119.0, 121.5, 126.7, 127.8, 130.3, 155.3, 165.1, 173.9. MS(ECI) m/z 485 and 487 (M+1).

### Example 43

### 4-[(5-Chloro-1H-benzimidazol-2-yl)thio]6-(hydroxymethyl) 1,3-dihydro-2H-benzimidazol-2-one

TBTU (104 mg, 0.32 mmol) and Hünig's base (57 μl, 0.32 mmol) were added to a solution of 7-[(5-chloro-1H-benzimidazol-2-yl)thio]-2-oxo-2,3-dihydro-1H-benzimidazole-5-carboxylic acid (100 mg, 0.28 mmol) in DMF (3 ml) at ambient temperature. After 10 min the reaction mixture was diluted with methanol. Then NABHI (20 mg, 0.54 mmol) was added and the reaction mixture was stirred for 1.5 h. Ethyl acetate and 0.5 M HCl were added and the layers separated. The aqueous phase was washed with ethyl acetate. The combined organic phases were washed with saturated NaHCO₃ (aq), dried (MgSO₄), filtered and concentrated. The crude mixture was purified on a Prep-HPLC to give 27 mg (29%) of the title compound. ¹H NMR (DMSO-d6): δ 4.46 (2H, s), 7.02 (1H, s), 7.06 (1H, d), 7.11 (1H, dd), 7.39 (1H, d), 7.45 (1H, s), 10.84 (1H, s). ¹³C NMR (DMSO-d6): δ 62.6, 105.9, 108.6, 121.5, 124.9, 125.8, 130.5, 131.9, 136.1, 155.3, 162.3. MS (ECI) m/z 347 and 349 (M+1).

Pharmacological Evaluation of Compounds

JNK-1 Kinase Filter Assay

Compounds were tested for inhibition of JNK-1 using a kinase filter assay. The test compounds were dissolved to 10 mM in dimethylsulphoxide (DMSO). The compounds were then diluted in DMSO using a half log dilution series. Diluted compounds were then further diluted 1 in 10 in kinase buffer (50 mM MOPS, pH 7.2 containing 0.1% (v/v) β-mercaptoethanol) to give 10 times the final concentration of compound in kinase buffer plus 10% (v/v) DMSO. 5 μl of each compound dilution was added to wells of a 96 well plate in duplicate. 5 μl of kinase buffer plus 10% (v/v) DMSO was added instead of compound to control wells (0% inhibition) and background wells (100% inhibition). 25 μl 15% (w/v) trichloroacetic acid (TCA) containing 100 mM ATP was added to background wells (100% inhibition).

20 μl of a mixture of ATF-2 protein substrate; magnesium chloride; unlabelled ATP; and ³³P-labelled ATP was added to each well such that the final concentration was 0.2 μM ATF-2; 10 mM magnesium chloride; 1 μM ATP and 0.1 μCi ³³P ATP. 25 μl of a mixture of JNK-1 (0.1 μg/well) was to start the reaction. The final reaction volume was 50 μl.

The kinase reactions were incubated at 21° C. for 60 min and the reaction stopped by precipitating the protein by the addition of 25 μl of 15% (w/v) TCA containing 100 mM ATP. The precipitate was allowed to form for 10 min and then filtered onto a GF/C unifilter 96 well plate. Each filter was washed ten times with approximately 0.3 ml water. The filter plate was dried at 30-40° C. for 60 min, 25 μl scintillant was added to each well and the plate sealed and radioactivity counted on a Packard Topcount microplate scintillation counter.

The compounds exemplified herein were all active in the abovescreen at a sub-micromolar level.

List of Abbreviations

DMSO Dimethylsulphoxide
MOPS 3-[N-Morpholino]-propanesulfonic acid
JNK c-Jun N-terminal kinase
MAP Mitogen-activated protein
LPS Lipopolysaccharide
TNFα Cytokines tumour necrosis factor
IL1 Interleukin-1
ATF Activating transcription factor
ATP Adenosine triphosphate
Th1 Type 1 T helper
MMP Matrix metalloproteinase
TCA Trichloroacetic acid
HOBT 1-Hydroxybenzotriazole hydrate
TBTU O-(1H-Benzotriazol-1-yl)N,N,N′,N′-tetramethyluronium tetrafluoroborate
HATU O-(7-Azabenzotriazol-1-yl)-N,N,N′,N′-tetramethyluronium hexafluorophosphate
Hünig's base NN-Diisopropylethylamine
DMF Dimethyl formamide
THF Tetrahydrofuran
M.pt. Meltpoint

1. A compound of formula (I) 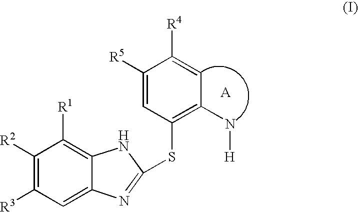
in the form of the free base, a salt thereof, a solvate thereof or a solvate of a salt thereof
wherein:
R¹ is hydrogen, CHCHR⁶, CCR⁶, CO₂R⁷, NHCOR⁷, CN or halogen;
R² is hydrogen, halogen, CN, OC₁₋₆ alkyl, or a 5- or 6-membered heterocyclic ring containing 1, 2 or 3 heteroatoms selected independently from N, O and S;
R³ is hydrogen or halogen;
R⁴ is hydrogen, OH, NH₂, NO₂ or NHR⁸;
R⁵ is hydrogen, COR⁹, CHO, CH₂OR¹⁰, OH, OC₁₋₆ alkyl, NH₂, NR¹⁰R¹¹, NHCONR¹⁰R¹¹, NHCOR¹⁰, CONR¹²R¹³, CONHR⁷, R⁷, or
R⁴ and R⁵ together with the carbon atoms to which they are attached form a 5-, 6- or 7-membered lactam ring;
R⁶ is C₁₋₆alkyl, substituted with 1, 2 or 3 substituents selected independently from hydroxy, OC₁₋₆ alkyl, OC₁₋₆ alkylOH and NR¹²R¹³;
R⁷ is CONHOC₁₋₆alkylOH, or C₁₋₆ alkyl optionally substituted with 1, 2, 3 or 4 substituents selected independently from hydroxy, OC₁₋₆alkyl and NR¹²R¹³, or
R⁷ is phenyl or a 5- or 6-membered heterocyclic ring containing 1, 2, 3 or 4 heteroatoms selected independently from N, O and S;
R⁸ is COR¹⁴ or (CH₂)ₙQ where n is 1, 2 or 3 and Q is NR¹⁵R¹⁶ or a 5-, 6- or 7-membered saturated heterocyclic ring containing 1, 2 or 3 heteroatoms selected independently from N, O and S, at least one of which must be N, with the exception that n can only be 1 when Q is a heterocyclic ring not linked through N;
R⁹ is hydroxy or OC₁₋₆ alkyl, which may be substituted with a 5- or 6-membered heterocyclic ring containing 1, 2 or 3 heteroatoms selected independently from N, O and S;
R¹⁰ and R¹¹ are selected independently from hydrogen and C₁₋₆ alkyl optionally substituted with 1, 2 or 3 substituents selected independently from hydroxy, OC₁₋₆ alkyl and NR¹²R¹³;
R¹² and R¹³ are selected independently from hydrogen and C₁₋₆ alkyl or R¹² and R¹³ form together a 5- or 6-membered heterocyclic ring containing 1, 2 or 3 heteroatoms selected independently from N, O and S and said ring may optionally be substituted with one or more substituents selected independently from hydroxy, oxo, C₁₋₆alkyl, OC₁₋₆ alkyl and C₁₋₆ alkylOH;
R¹⁴ is hydrogen or C₁₋₆ alkyl;
R¹⁵ and R¹⁶ are selected independently from hydrogen and C₁₋₆ alkyl;
A is a 5- or 6-membered saturated or a 5- or 6-membered aromatic ring optionally containing one or more heteroatoms selected independently from N, O and S, containing one or more C═O groups, and said ring may optionally be substituted with one or more substituents selected independently from C₁₋₆ alkyl, SO₂C₁₋₆ alkyl, SOC₁₋₆ alkyl, CO₂H and CHO.

2. A compound according to claim 1, wherein:
R² is hydrogen, halogen, CN, or a 5- or 6-membered heterocyclic ring containing 1, 2 or 3 heteroatoms selected independently from N, O and S;
R⁵ is hydrogen, COR⁹, CHO, CH₂OR¹⁰, OH, OC₁₋₆ alkyl, NH₂, NR¹⁰R¹¹, NHCONR¹⁰R¹¹, NHCOR¹⁰ or
R⁴ and R⁵ together with the carbon atoms to which they are attached form a 5-, 6- or 7-membered lactam ring;
R⁶ is C₁₋₆ alkyl, substituted with 1, 2 or 3 substituents selected independently from hydroxy, OC₁₋₆ alkyl and NR¹² R¹³;
R⁹ is hydroxy or OC₁₋₆ alkyl; and
R¹² and R¹³ are selected independently from hydrogen and C₁₋₆ alkyl.

3. The compound according to claim 1, wherein R¹ is hydrogen, CN, CO₂CH₃, CO₂CH₂CH₃, CO₂CH₂CH₂OH, halogen or NHCOR⁷, wherein R⁷ is furyl or phenyl.

4. The compound according to claim 1, wherein R³ is hydrogen.

5. The compound according to claim 1, wherein R⁴ is hydrogen, NH₂, NO₂ or NHR⁸, wherein R⁸ is CH₂-pyrrolidine.

6. The compound according claim 5, wherein R⁴ is NHR⁸, wherein R⁸ is CH₂-piperidine.

7. The compound according to claim 1, wherein R⁵ is hydrogen or COR⁹, wherein R⁹ is hydroxy.

8. The compound according to claim 1, wherein R⁵ is CONR¹²R¹³, CONHR⁷ or R⁷.

9. The compound according to claim 1, wherein R⁷ is CONHOC₁₋₆alkylOH, or C₁₋₆ alkyl optionally substituted with hydroxy, or R⁷ is phenyl or a 5- or 6-membered heterocyclic ring containing 1, 2, 3 or 4 heteroatoms selected independently from N and O.

10. The compound according to claim 1, wherein group A, together with the phenyl group to which it is attached, forms a bicyclic group of formula (A) or (B): 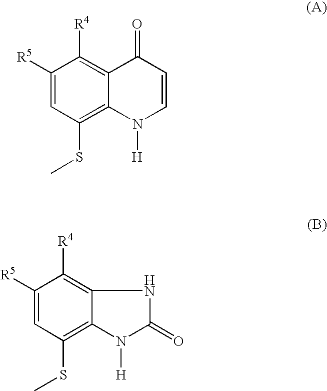
wherein R⁴ and R⁵ are as defined in claim 1.

11. A compound which is
8-(1H-benzimidazol-2-ylsulfanyl)-4(1H)-quinolinone,
8-[(5-chloro-1H-benzimidazol-2-yl)sulfanyl]-4(1H)-quinolinone,
2-(4-oxo-1,4-dihydro-quinolin-8-ylsulfanyl)-1H-benzoimidazole-4-carboxylic acid methyl ester,
8-[(5-chloro-1H-benzimidazol-2-yl)sulfanyl]-5-nitro-4(1H)-quinolinone,
5-amino-8-[(5-chloro-1H-benzimidazol-2-yl)sulfanyl]-4(1H)-quinolinone,
8-[(5-chloro-1H-benzimidazol-2-yl)sulfanyl]-5-{[(2R)-pyrrolidinylmethyl]amino}-4(1H)-quinolinone,
8-[(5-chloro-1H-benzimidazol-2-yl)sulfanyl]-5-[(4-piperidinylmethyl)amino]-4(1H)-quinolinone,
5-chloro-2-[(4-oxo-1,4-dihydroquinolin-8-yl)thio]-1H-benzimidazole-4-carbonitrile,
methyl 5-chloro-2-[(4-oxo-1,4-dihydroquinolin-8-yl)thio]-1H-benzimidazole-4-carboxylate,
N-{5-chloro-2-[(4-oxo-1,4-dihydroquinolin-8-yl)thio]-1H-benzimidazol-4-yl}benzamide,
N-{5-chloro-2-[(4-oxo-1,4-dihydroquinolin-8-yl)thio]-1H-benzimidazol-4-yl}-2-furamide,
4-[(5-chloro-1H-benzimidazol-2-yl)thio]-1,3-dihydro-2H-benzimidazol-2-one,
7-[(5-chloro-1H-benzimidazol-2-yl)thio]-2-oxo-2,3-dihydro-1H-benzimidazole-5-carboxylic acid, or
7-{[5-chloro-4-(methoxycarbonyl)-1H-benzimidazol-2-yl]thio}-2-oxo-2,3-dihydro-1H-benzimidazole-5-carboxylic acid
in the form of the base, a salt thereof, a solvate thereof or a solvate of a salt thereof.

12. A compound which is
7-({5-chloro-4-[(2-hydroxyethoxy)carbonyl]-1H-benzimidazol-2-yl}thio)-2-oxo-2,3-dihydro-1H-benzimidazole-5-carboxylic acid,
methyl 7-[(5-chloro-1H-benzimidazol-2-yl)thio]-2-oxo-2,3-dihydro-1H-benzimidazole-5-carboxylate,
ethyl 7-[(5-chloro-1H-benzimidazol-2-yl)thio]-2-oxo-2,3-dihydro-1H-benzimidazole-5-carboxylate,
2-morpholin-4-ylethyl 7-[(5-chloro-1H-benzimidazol-2-yl)thio]-2-oxo-2,3-dihydro-1H-benzimidazole-5-carboxylate,
7-[(5-chloro-6-fluoro-1H-benzimidazol-2-yl)thio]-2-oxo-2,3-dihydro-1H-benzimidazole-5-carboxylic acid,
7-[(5-methoxy-1H-benzimidazol-2-yl)thio]-2-oxo-2,3-dihydro-1H-benzimidazole-5-carboxylic acid,
7-[(5-bromo-1H-benzimidazol-2-yl)thio]-2-oxo-2,3-dihydro-1H-benzimidazole-5-carboxylic acid,
7-[(4-bromo-6-fluoro-1H-benzimidazol-2-yl)thio]-2-oxo-2,3-dihydro-1H-benzimidazole-5-carboxylic acid,
7-[(5-chloro-1H-benzimidazol-2-yl)thio]-N-[2-(4-morpholin-4-ylethyl]-2-oxo-2,3-dihydro-1H-benzimidazole-5-carboxamide,
7-[(5-chloro-1H-benzimidazol-2-yl) thio]-N-[3-dimethylamino) ethyl]-2-oxo-2,3-dihydro-1H-benzimidazole-5-carboxamide,
4-[(5-chloro-1H-benzimidazol-2-yl) thio]-6-[(4-methylpiperazin-1-yl) carbonyl]-1,3-dihydro-2H-benzimidazol-2-one,
7-[(5-chloro-1H-benzimidazol-2-yl)thio]-N-methyl-2-oxo-2,3-dihydro-1H-benzimidazole-5-carboxamide,
7-[(5-chloro-1H-benzimidazol-2-yl)thio]-N-(2-hydroxyethyl)-2-oxo-2,3-dihydro-1H-benzimidazole-5-carboxamide,
4-[(5-chloro-1H-benzimidazol-2-yl)thio]-6-(piperazin-1-ylcarbonyl)-1,3-dihydro-2H-benzimidazol-2-one dihydrochloride,
7-[(5-chloro-1H-benzimidazol-2-yl) thio]-2-oxo-2,3-dihydro-1H-benzimidazole-5-carboxamide,
7-[(5-chloro-1H-benzimidazol-2-yl) thio]-N-[2-(2-hydroxyethoxy) ethyl]-2-oxo-2,3-dihydro-1H-benzimidazole-5-carboxamide,
4-[(5-chloro-1H-benzimidazol-2-yl) thio]-6-{[4-(2-hydroxyethyl) piperazin-1-yl]carbonyl}-1,3-dihydro-2H-benzimidazol-2-one,
7-[(5-chloro-1H-benzimidazol-2-yl) thio]-2-oxo-N-4-piperidin-4-yl-2,3-dihydro-1H-benzimidazole-5-carboxamide dihydrochloride,
N-(3-aminopropyl)-7-[(5-chloro-1H-benzimidazol-2-yl)thio]-2-oxo-2,3-dihydro-1H-benzimidazole-5-carboxamide dihydrochloride,
7-[(5-chloro-1H-benzimidazol-2-yl)]thio-2-oxo-N-[3-(2-oxopyrrolidin-1-yl) propyl]-2,3-dihydro-1H-benzimidazole-5-carboxamide, or
4-[(5-chloro-1H-benzimidazol-2-yl)thio]-6-(hydroxymethyl) 1,3-dihydro-2H-benzimidazol-2-one,
in the form of the base, a salt thereof, a solvate thereof or a solvate of a salt thereof.

13. A compound of formula (I) 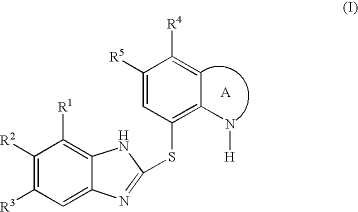
wherein:
R¹ is hydrogen, CO₂R⁷, NHCOR⁷ or halogen;
R² is hydrogen or halogen;
R³ is hydrogen or halogen;
R⁴ is hydrogen, NH₂ or NO₂;
R⁵ is hydrogen;
R⁷ is C₁₋₆alkyl optionally substituted with 1, 2 or 3 substituents selected independently from hydroxy;
A is a 5-membered aromatic ring containing one or more heteroatoms selected independently from N and O; and said ring may optionally be substituted with one or more substituents selected independently from C₁₋₆ alkyl, SO₂C₁₋₆ alkyl, SOC₁₋₆ alkyl and CHO,
in the form of the base, a salt thereof, a solvate thereof or a solvate of a salt thereof.

14. A compound according to claim 13, wherein group A, together with the phenyl group to which it is attached, forms a bicyclic group of formula (C): 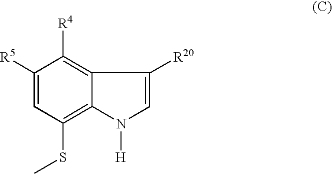
wherein R⁴ and R⁵ are as defined in claim 13 and R²⁰ is SOCH₃, SO₂CH₃, CH₃, CO₂H or CHO.

15. A compound, which is
7-[(5-chloro-1H-benzimidazol-2-yl)thio]-3-(methylsulfonyl)-1H-indol-4-amine,
methyl 5-chloro-2-{[3-(methylsulfonyl)-1H-indol-7-yl]thio}-1H-benzimidazole-4-carboxylate,
5-chloro-2-[(3-methyl-4-nitro-1H-indol-7-yl)thio]-1H-benzimidazole,
7-[(5-chloro-1H-benzimidazol-2-yl)thio]-3-methyl-1H-indol-4-amine,
5-chloro-2-{[3-(methylsulfinyl)-4-nitro-1H-indol-7-yl]thio}-1H-benzimidazole,
7-[(5-chloro-1H-benzimidazol-2-yl)thio]-3-(methylsulfinyl)-1H-indol-4-amine,
4-amino-7-[(5-chloro-1H-benzimidazol-2-yl)thio]-1H-indole-3-carbaldehyde, or
7-[(5-chloro-1H-benzimidazol-2-yl)thio]-3-(methylsulfonyl)-1H-indole,
in the form of the base, a salt thereof, a solvate thereof or a solvate of a salt thereof.

16. The compounds according to claim 1 in the form of a pharmaceutically acceptable salt.

17. A process for the preparation of a compound as defined in claim 1 or 13 comprising
(a) reacting a compound of formula (II): 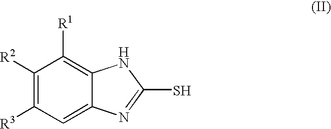
wherein R¹, R² and R³ are as defined in formula (I) with a compound of formula (III): 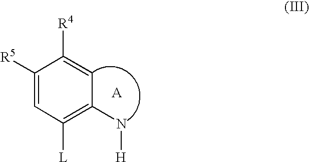
wherein A, R⁴ and R⁵ are as defined in formula (I) and L is a leaving group, or
(b) reacting a compound of formula (IV): 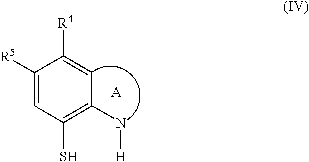
wherein A, R⁴ and R⁵ are as defined in formula (I) with a compound of formula (V): 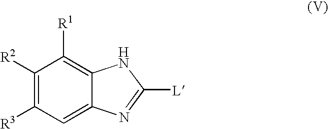
wherein R¹, R² and R³ are as defined in formula (I) and L′ is a leaving group.

18. A process for the preparation of a compound as defined in claim 1 comprising converting a compound of formula (VI), wherein R⁵ is carboxy and R¹, R², R³ and R⁴ are as defined in claim 1, to a compound of formula (Ia), wherein R¹, R², R³, R⁴ and R⁵ are as defined in claim 1,

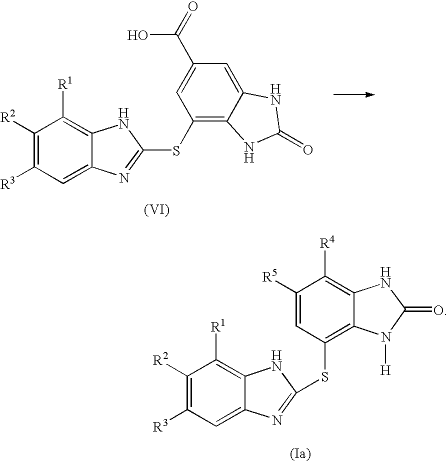

19-22. (Canceled)

23. A method for treatment of JNK mediated conditions, comprising administering to a patient in need of such treatment a therapeutically effective amount of the compound as defined in claim 1.

24. A method for treatment of Alzheimer's disease, Parkinson's disease, ALS, epilepsy and seizures, Huntington's disease, traumatic brain injury or haemorrhaging stroke, comprising administering to a patient in need of such treatment a therapeutically effective amount of the compound as defined in claim 1.

25. The method according to claim 24, for treatment of Alzheimer's disease.

26. A pharmaceutical composition comprising as active ingredient a therapeutically effective amount of the compound as defined in claim 1, in association with pharmaceutically acceptable excipients, diluents and/or inert carriers.

27-29. (Canceled)

30. A compound of formula (VI) 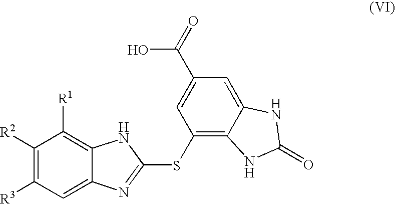
wherein:
R¹ is hydrogen, CO₂R⁷, NHCOR⁷, CN or halogen;
R² is hydrogen, halogen, OC₁₋₆ alkyl, or a 5- or 6-membered heterocyclic ring containing 1, 2 or 3 heteroatoms selected independently from N, O and S; and
R³ is hydrogen or halogen;
R⁶ is C₁₋₆ alkyl, substituted with 1, 2 or 3 substituents selected independently from hydroxy, OC₁₋₆ alkyl, OC₁₋₆ alkylOH and NR¹²R¹³;
R⁷ is CONHOC₁₋₆alkylOH, or C₁₋₆ alkyl optionally substituted with 1, 2, 3 or 4 substituents selected independently from hydroxy, OC₁₋₆alkyl and NR¹²R¹³, or
R⁷ is phenyl or a 5- or 6-membered heterocyclic ring containing 1, 2, 3 or 4 heteroatoms selected independently from N, O and S,
in the form of the base, a salt thereof, a solvate thereof or a solvate of a salt thereof.

31. (Canceled)
